(19) United States
(12) Patent Application Publication
(10) Pub. No.: US 2010/0159540 A1
(43) Pub. Date: Jun. 24, 2010

(54) SYNTHESIS OF RESOLVINS AND INTERMEDIATES, COMPOUNDS PREPARED THEREBY, AND USES THEREOF

(76) Inventors: Ana Rodriguez, Marlton, NJ (US); Bernd Spur, Marlton, NJ (US)

Correspondence Address: KLAUBER & JACKSON, 411 HACKENSACK AVENUE, HACKENSACK, NJ, 07601, US

(21) Appl. No.: 12/450,422

(22) PCT Filed: Mar. 26, 2008
(86) PCT No.: PCT/US08/03943

§ 371 Date: Mar. 3, 2010

Related U.S. Application Data
(60) Provisional application No. 60/920,112, filed on Mar. 26, 2007.

(51) Int. Cl. C12P 7/64; C07C 51/367; C07C 59/42; C07D 309/30

(52) U.S. Cl. 435/134; 554/126; 554/219; 549/323

(57) ABSTRACT

Methods are disclosed for the preparation of a new class of lipid mediators known as resolvins, with Resolvin D6 (4,17-dihydroxy-5E,7Z,10Z,13Z,15E,19Z-docosahexaenoic acid) being exemplary. Also disclosed are methods for the efficient synthesis of key intermediates in the preparation of such resolvins, such as isotopically labeled ω-3 fatty acid metabolites, and derivatives and analogs thereof. The invention likewise extends to the intermediates so prepared, and to the resolvins prepared with their use.

### CROSS REFERENCE TO RELATED APPLICATIONS

The present application is a National Stage Application claiming the priority of co-pending PCT Application No. PCT/US2008/003943 filed Mar. 26, 2008, which in turn, claims priority from U.S. Provisional Application Ser. No. 60/920,112, filed Mar. 26, 2007. Applicants claim the benefits of 35 U.S.C. §120 as to the PCT application and priority under 35 U.S.C. §119 as to the said U.S. Provisional application, and the entire disclosures of both applications are incorporated herein by reference in their entireties.

### BACKGROUND

Recently, Serhan et al. reported a new class of lipid mediators derived from docosahexaenoic and eicosapentaenoic acid that posses potent anti-inflammatory and immunoregulatory activities in the low picomolar to nanomolar range.¹⁻³⁵ These new compounds are formed in vivo via cell-cell interaction and were named Resolvins (resolution phase interaction products). Docosahexaenoic acid is highly enriched in brain, synapses and retina. Deficiencies of this ω-3 fatty acid are associated with autism, Alzheimer's disease, stroke, hyperactivity, schizophrenia and peroxisomal disorders.

Other diseases that are associated with diminished formation of these “good lipid mediators” are asthma, kidney diseases, inflammatory bowel disease, rheumatoid arthritis, sepsis and other neutrophil-driven diseases. Serhan's work has established for the first time the molecular basis and the mechanism of the immune protective action conferred by ω-3 fatty acids. Since from natural sources only tiny amounts are available they have to be prepared by total chemical synthesis in order to expedite continuing biological and pharmacological investigations.^{36,37} These natural products could be novel lead structures for the development of drugs that inhibit PMN infiltration at the site of inflammation and to circumvent side effects of current anti-inflammatory drugs. FIG. 1 shows the conversion of DHA into Resolvins and Neuroprostanes.

### SUMMARY OF THE INVENTION

The invention pertains to methods to prepare Resolvin D6 (4,17-dihydroxy-5E,7Z,10Z,13Z,15E,19Z-docosahexaenoic acid). Methods are disclosed which provide for the production of Resolvin D6 which is enriched in the (R)-configuration at the C4 hydroxyl. Methods are also disclosed which provide for the production of Resolvin D6 which is enriched in the (S)-configuration at the C4 hydroxyl. Methods are also disclosed which provide for the production of Resolvin D6 which is enriched in the (S)-configuration of the C17 hydroxyl. Several approaches to the efficient synthesis of Resolvin D6, key intermediates in the synthesis of Resolvin D6, derivatives of Resolvin D6, and analogs of Resolvin D6 are disclosed. It should be recognized that chemical transformations, process optimizations, and purification strategies disclosed herein can be utilized by one of ordinary skill to prepare structural analogs and derivatives of Resolvin D6 not specifically identified herein, but which may have utility and are within the scope of the present invention.

The invention also pertains to a process in which systematic investigations have revealed a surprising and very useful improvement in catalytic asymmetric transfer hydrogenation of carbonyl compounds to chiral alcohols possessing a very high degree of enantiomeric excess(ee). The improvement comprises methodology which magnifies the efficiency of asymmetric transfer hydrogenation catalysts as much as one thousand times over the methods currently in use. The improvement further comprises techniques which allow for extremely mild conditions in the preparation of sensitive hydroxyl-containing molecules.

The invention also pertains to methods for the preparation of isotopically labeled ω-3 fatty acid metabolites. Disclosed is the preparation of d-4-7(S),17(S)-Resolvin D5 (10,11,13,14-tetradeutero-7(S),17(S),4Z,9E,10Z,13Z,15E,10Z docosahexaenoic acid). The methodology disclosed herein is generally applicable to the preparation of several other isotopically enriched metabolites of unsaturated biomolecules including, but not limited to, arachidonic acid, eicosapentaenoic acid, and docosahexaenoic acid. Such isotopically enriched metabolites are considered to be within the scope of the invention. It will be appreciated that the disclosed method may be utilized by one of ordinary skill to provide a large variety of deuterated or tritiated compounds.

The invention further pertains to methods to prepare key intermediates in the syntheses of resolvins utilizing inexpensive chiral starting materials from natural sources. The disclosed methods allow the large-scale preparation of optically pure intermediates useful in the synthesis of several resolvins.

### BRIEF DESCRIPTION OF THE DRAWING

FIG. 1 is a flow diagram depicting the in vivo disposition of DHA and the formation of Resolvins and Neuroprostanes.

### DETAILED DESCRIPTION OF THE PREFERRED EMBODIMENTS

The following terms are intended to have the meanings presented therewith below and are useful in understanding the description and intended scope of the present invention.

When describing the compounds, pharmaceutical compositions containing such compounds and methods of using such compounds and compositions, the following terms have the following meanings unless otherwise indicated. It should be further understood that the terms “groups” and “radicals” can be considered interchangeable when used herein.

The articles “a” and “an” may be used herein to refer to one or to more than one (i.e. at least one) of the grammatical objects of the article. By way of example “an analogue” means one analogue or more than one analogue.

“Acyl” refers to a radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cyclohexylcarbonyl, cyclohexylmethylcarbonyl, benzoyl, benzylcarbonyl and the like.

“Alkyl” refers to monovalent saturated alkane radical groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms.

“Substituted alkyl” refers to those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable vehicle” refers to a diluent, adjuvant, excipient or carrier with which a compound of the invention is administered.

“Pharmaceutically acceptable salt” refers to the non-toxic, inorganic and organic acid addition salts, and base addition salts, of compounds of the present invention, in particular they are pharmaceutically acceptable and possess the desired pharmacological activity of the parent compound. These salts can be prepared in situ during the final isolation and purification of compounds useful in the present invention. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. Conventional solvents include water, ethanol, acetic acid and the like, therefore, representative solvates include hydrates, ethanolates and methanolates.

“Subject” refers to humans and non-human mammals. In certain embodiments, a subject is a human.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, aryl, C₇-C₁₂ substituted aryl, and C₇-C₁₂ arylalkyl esters of the compounds of the invention.

“Isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon 13 (¹³C), nitrogen-15 (¹⁵N), or the like. It will be understood that, in a compound where such isotopic substitution is made, the following atoms, where present, may vary, so that for example, any hydrogen may be 2H/D, any carbon may be ¹³C, or any nitrogen may, be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Calm and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

“Subject” includes humans. The terms “human”, “patient” and “subject” are used interchangeably herein.

“Prophylaxis” means a measure taken for the prevention of a disease.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means that amount of a drug or pharmaceutical agent that will elicit the biological or medical response of a subject that is being sought by a medical doctor or other clinician. The “therapeutically effective amount” can vary depending on the compound, the disease and its severity, and the age, weight, etc., of the subject to be treated.

### DETAILED DESCRIPTION OF THE INVENTION

This invention relates to the first total synthesis of 4(R,S), 17(S)-Resolvin D6 (1). As shown in the retrosynthetic scheme (Scheme 1), the chiral center at C-17 was obtained via an enzymatic reaction with 15-LOX from Soya beans³⁸ whereas the hydroxyl group at C-4 arises from an iodolactonization-elimination sequence.³⁹⁻⁴³

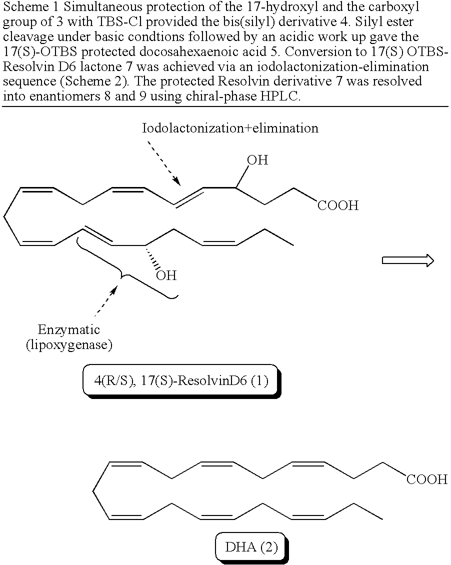

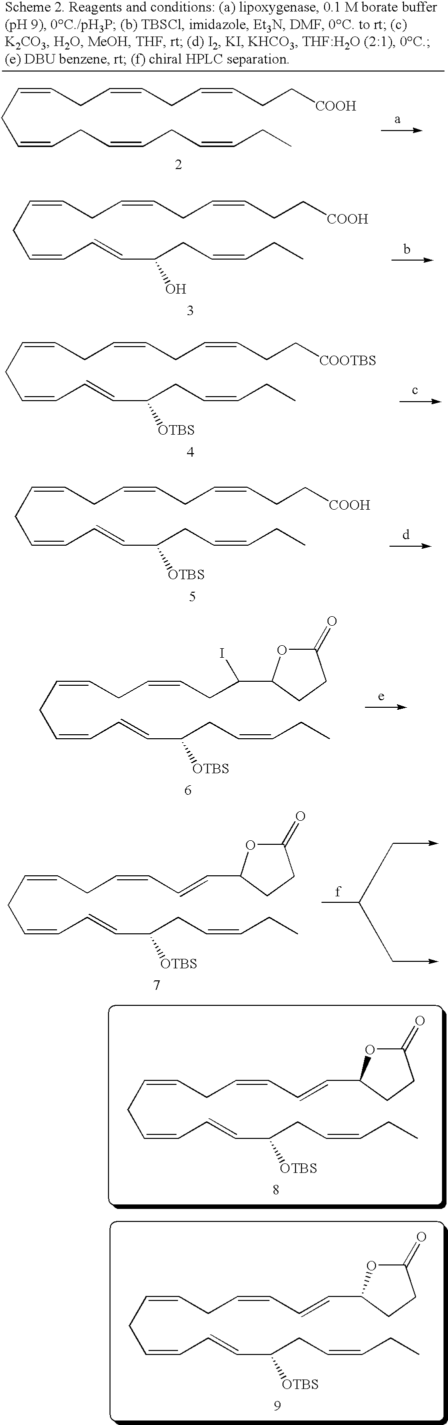

A short synthesis of Resolvin D6 14 from docosahexaenoic acid is outlined in Scheme 3, below. The C-4 epimers can be easily separated by chiral HPLC giving access to the 4(S), 17(S) Resolvin D6 14 and the 4(R), 17(S) Resolvin D6 15. As seen in Scheme 3, no manipulation of protecting groups was necessary. The enzymatic reaction with 15-LOX gives after triphenylphosphine reduction of the intermediate C17 hydroperoxide at pH 9 the 17(S) hydroxy-docosahexaenoic acid 3 in 70% yield after flash chromatography. Iodolactonization with KJ/J2 followed by HJ elimination with 1,8-Diazabicyclo[5.4.0]undec-7-ene in benzene at room temperature produced 4(R,S),17(S)-Resolvin D6-lactone 11 in 60% yield.

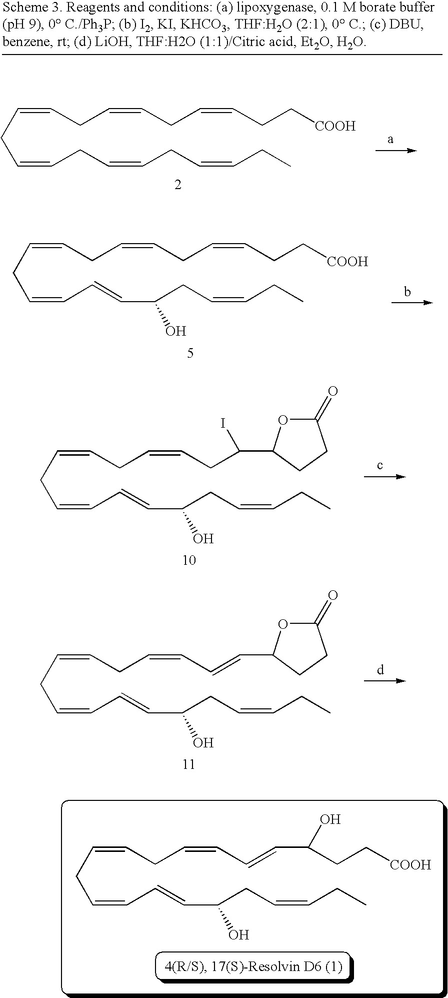

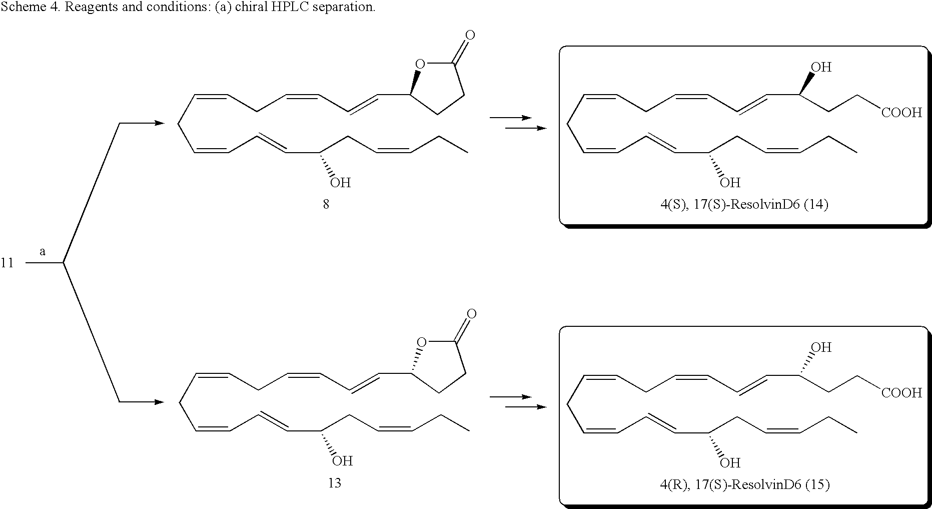

As shown in Scheme 4 the products 12 and 13 can be easily separated by chiral HPLC. Mild ester hydrolysis with lithium hydroxide followed by gentile citric acid neutralization provided 4(S),17(S)-Resolvin D6 14 and 4(R),17(S)-Resolvin D6 15.

In order to generate directly the chiral center in the 4-position we explored the chiral iodolactonization of docosahexaenoic acid. The asymmetric iodolactonization for unsubstituted unsaturated fatty acids produced the iodolactones with only very low enantiomeric excesses.⁴³⁻⁵²

Using Myers' pseudoephedrine amide⁵³ as a chiral auxiliary we were able to obtain the chiral iodolactone from docosahexaenoic acid with 50% ee directly (Scheme 5). This product was converted into 4-(S)-Zooxanthellactone 18.⁵⁴ Lactone 18 was converted into Resolvin D6 14 via the enzymatic 15-LOX reaction followed by lactone hydrolysis.

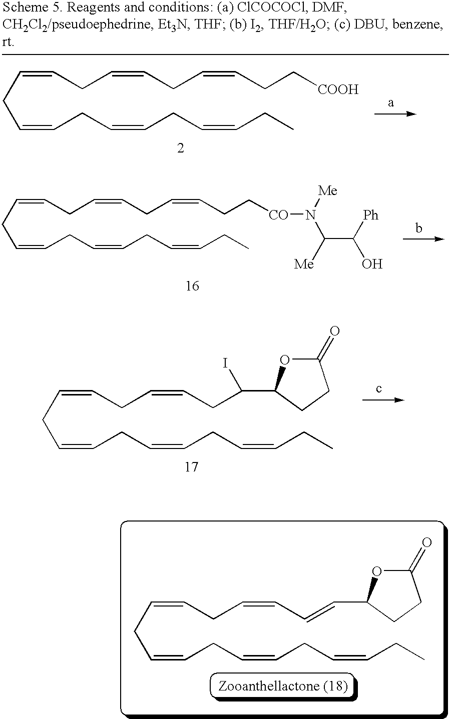

An alternative route was developed starting from the 4-hydroxydocosahexaenoic acid-γ-lactone as outlined in Scheme 6. Opening of the lactone 20 with LiOH in THF/H₂O followed by mild acidification with citric acid in the presence of ether furnished the hydroxy acid 21 that was converted to the hydroxy methyl ester 22 in quantitative yield upon treatment with diazomethane. Alternatively, opening of the lactone 20 with methanol/triethylamine containing a trace of water furnished the 4(R,S)-hydroxy methyl ester 22. Mild oxidation with the Dess-Martin periodinane reagent at room temperature gave the conjugated di-unsaturated ketone 23 with retention of the double bond geometry. The asymmetric reduction⁵⁴⁻⁶³ of 23 with various reducing agents⁶⁴⁻⁷³ is summarized in Table 1.

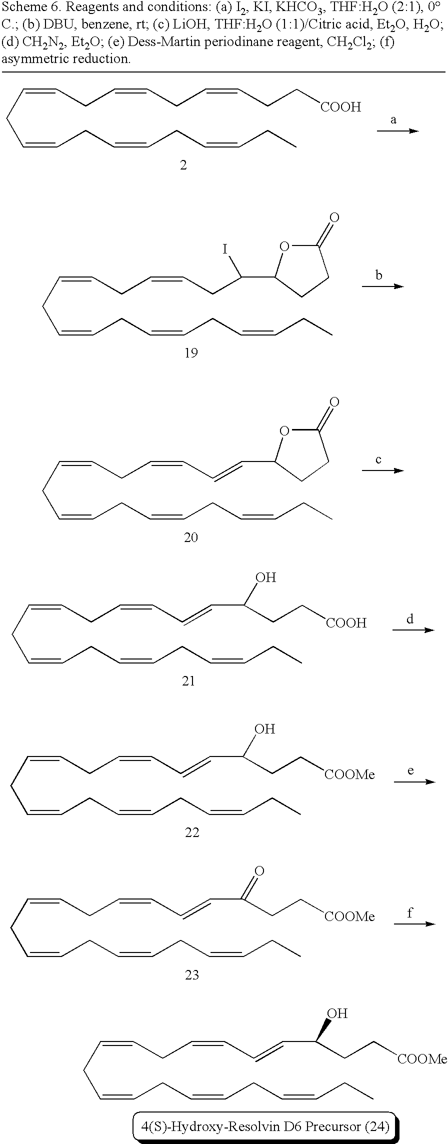

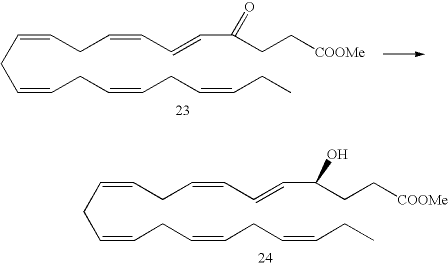

TABLE 1

| REDUCTION TYPE | REAGENTS | ee |
|---|---|---|
| “Corey-chiral-reduction” | (R)-2-methyl-CBS-oxazaborolidine, | 26% |
| | BH₃•Me₂S, toluene | |
| “Corey-chiral-reduction” | (R)-2-methyl-CBS-oxazaborolidine, | 57% |
| | catecholborane, CH₂Cl₂ | |
| Transfer hydrogenation | “Ru-TsDPEN”-cat, ⁱPrOH | 26% |
| “Noyori-chiral-reduction” | BINAL-H, THF | 12% |

Total Synthesis of d₄-Resolvin D5

The synthesis of deuterated Resolvins is required for the accurate determination of the in vivo production of Resolvins in humans by the isotope dilution method. In the following paragraph we describe a short total synthesis of the d₄-Resolvin D5 33 using the same strategy as published in our first total synthesis of Resolvin D5.³⁷ In this synthesis both chiral hydroxyl-groups were protected as TBS ethers. Silyl-cleavage of the di-acetylene-precursor 30 was cleanly achieved with a catalytic amount of acetyl chloride in methanol to give 31. Introduction of the deuterium without isomerization or over-reduction of the double bonds was achieved with Zn (Cu/Ag)³⁷ in 1:1 CD₃OD/D₂O at 40° C. for 5 h to give 32 in 70% yield after HPLC purification. Mild alkaline hydrolysis of d₄-7(S),17(S)-Resolvin D5 methyl ester (32) with 1N LiOH in THF at 0° C. followed by acidification with NaH₂PO₄ in the presence of EtOAc gave d₄-7(S),17(S)-Resolvin D5 33 (Scheme 7).

Synthesis of d₄-Resolvin D5

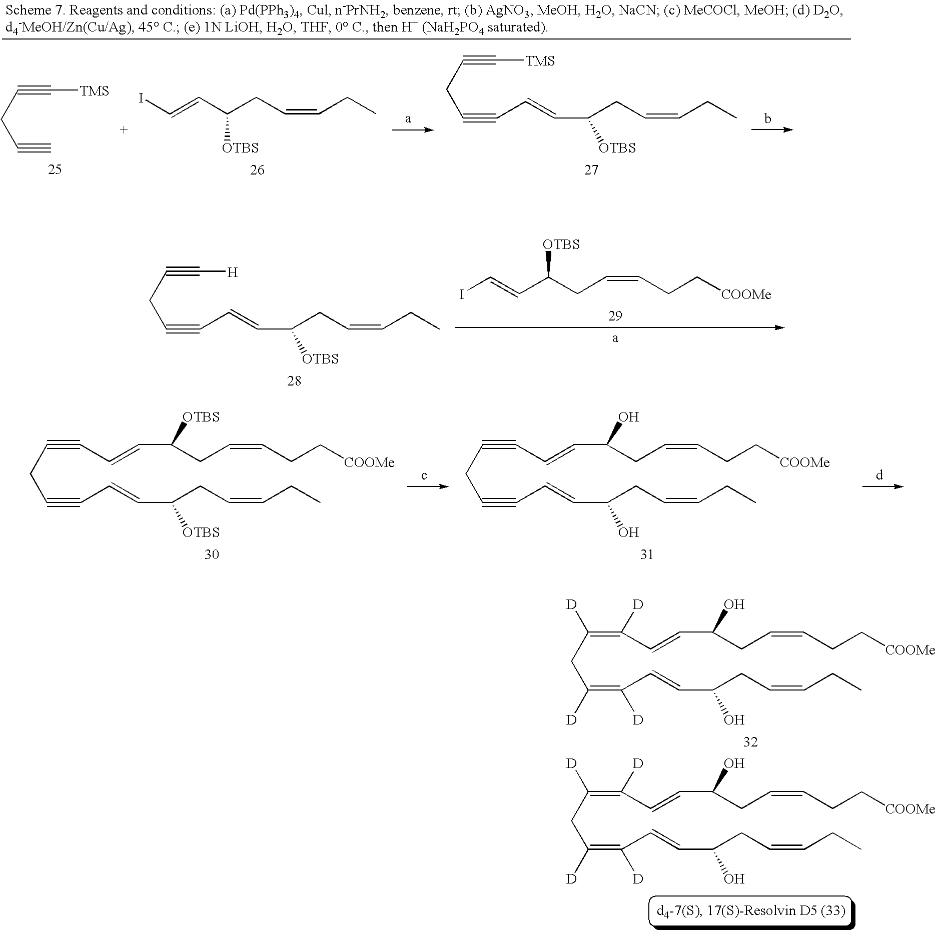

### Improved Synthesis of Key Intermediates of Resolvin E and D and Other Eicosanoids

For the synthesis of larger quantities of the different Resolvins using our previously reported synthesis, novel approaches to the key chiral intermediates have been developed. This methodology is amenable to large scale preparations, and is useful for the synthesis of many bioactive metabolites, including lipoxins, D-series resolvins (derived from docosahexaenoic acid, E-series resolvins (derived from eicosapentaenoic acid), neuroprotectins, isoprostanes, phytoprostanes and neuroprostanes as well as hydroxyeicosatetraenoic acids (HETEs) and leukotrienes. As outlined in Scheme 8 the selective mono-acylation of bis-trimethylsilyl acetylene with various acid chlorides was achieved in high yield using a catalytic amount (5%) of InBr₃.⁷⁴ This is an improvement over preparations of trimethylsilyl alpha-acetylenic ketones described in the literature in which an equimolar quantity of AlCl₃ is used.

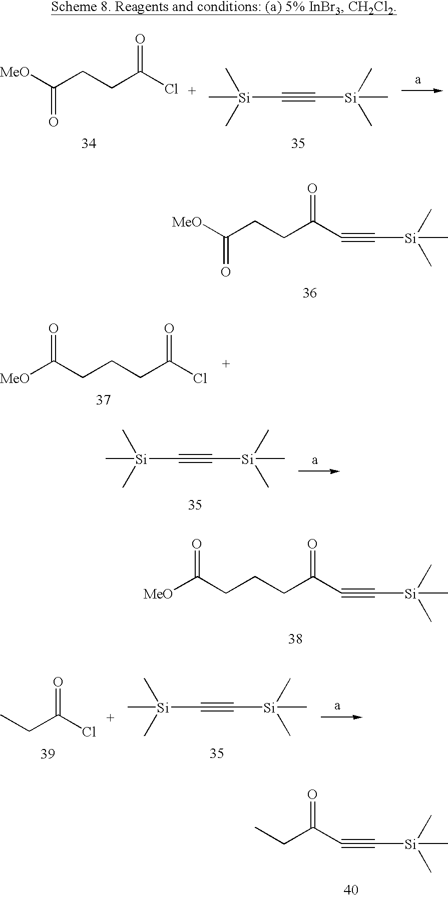

The Noyori asymmetric transfer hydrogenation⁶⁴⁻⁶⁷ produced the chiral TMS-acetylenic alcohols with >99% ee. In contrast to published work using 5-10% of the Noyori catalyst ((S,S)-TsDPEN)Ru(p-cymene)Cl₂ (turn over numbers 10-20) we found that a modification of the procedure gives turn over numbers>10,000. The freshly prepared catalyst (0.01%) was added to degassed isopropanol under argon and aged for 20 min, and then the TMS-acetylenic ketone in isopropanol was slowly added over several hours. The reaction mixture was stirred for additional 12 hours and, after evaporation of the solvent, the product was purified by flash chromatography to give the chiral alcohol 41 in >94% yield with 99% ee (Scheme 9). Subsequent protection of the newly-formed alcohol as a TBS ether, followed by unmasking of the terminal acetylene by selective desilylation, reduction to the vinyl tin derivative, and finally tin-halogen exchange furnished vinyl bromide 45. In like fashion, acetylenic ketones 38 and 40 provided important intermediates 46 and 47.

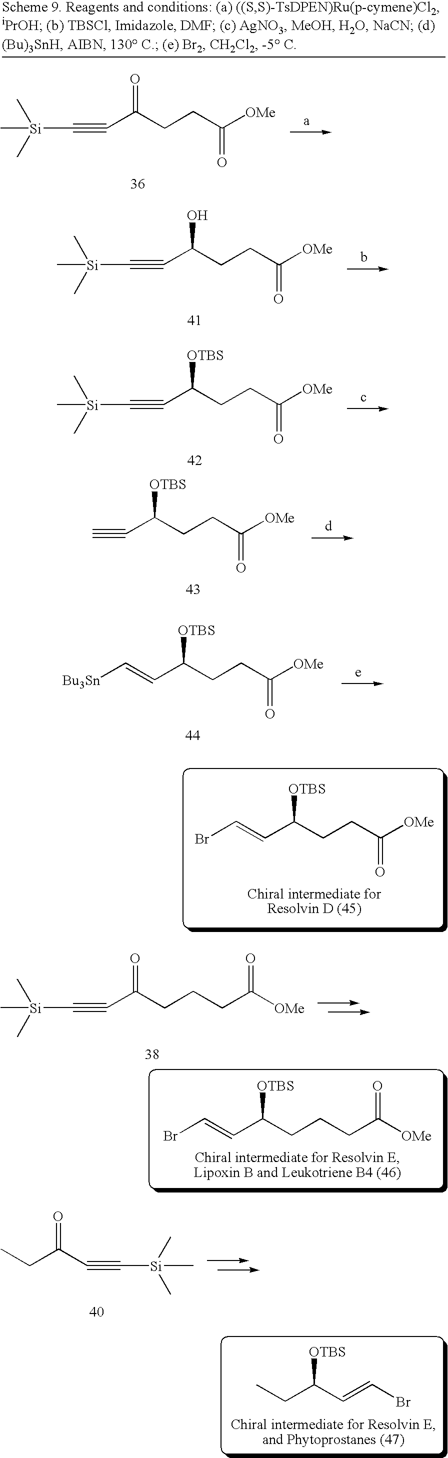

Synthesis of Resolvins from Malic Acid

A new general synthesis was developed starting from commercial (S)-malic acid. Esterification and reduction with LAH gives optical pure 1,2(S),4-butanetriol 48. Reaction with acetone in the presence of a catalytic amount TsOH at room temperature gives the 5-membered acetonide 49 containing a small amount of 6-membered acetonide 50. The crude mixture was converted into the mixture of p-toluenesulfonates (only 51 is shown). As described by Taber et al.,⁷⁴ on exposure to NaJ under Finkelstein conditions in the presence of a small amount of copper, 51 produced cleanly the required chiral iodide 52. Reaction of 52 with 1.2 equivalents of triphenylphosphine in acetonitrile under reflux in the presence of NaHCO₃ provided the chiral phosphonium iodide 53 in quantitative yield. The crystalline phosphonium iodide 53 was free of any isomeric impurities and was identical in all respects, including NMR and optical rotation, with material previously prepared by a rather lengthy route. This sequence could be run on the 0.5 molar scale without any difficulties (Scheme 10).

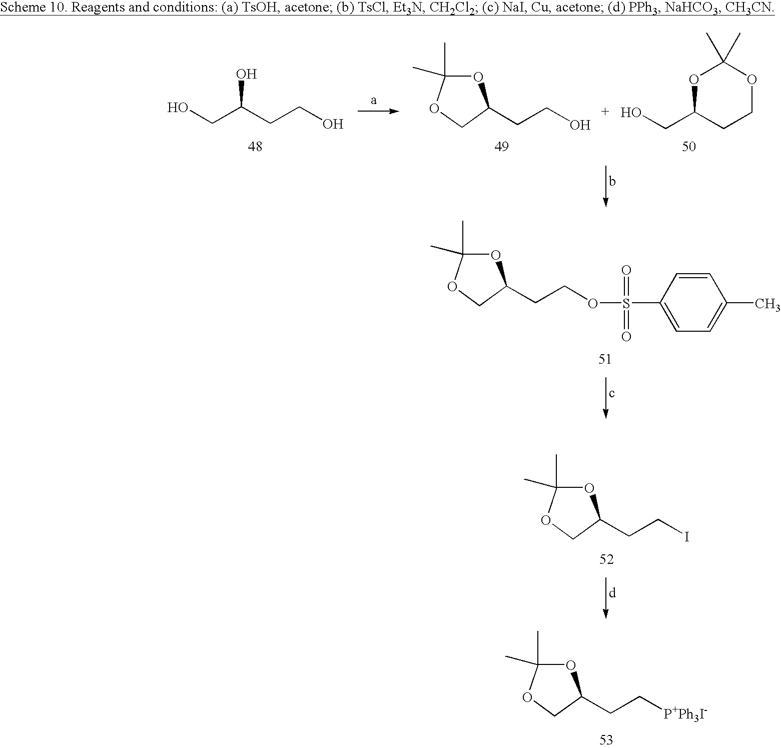

The ylide of 53 was generated in THF at −78 C using potassium bis(trimethylsilyl)amide. Reaction with the aldehydes 54 and 57 produced cleanly the chiral isopropylidene derivatives 55 and 58, respectively. Cleavage of the isopropylidene groups with 1N HCl in methanol at 0 C gave the chiral diols in quantitative yield. Protection with TES-Cl gave the di-TES derivatives 56 and 59 in quantitative yield that were identical with the material we prepared in our first total synthesis of Resolvin D2 and D5^{36,37}. This sequence has been performed in multi-gram quantities in the laboratory. (Scheme 11)

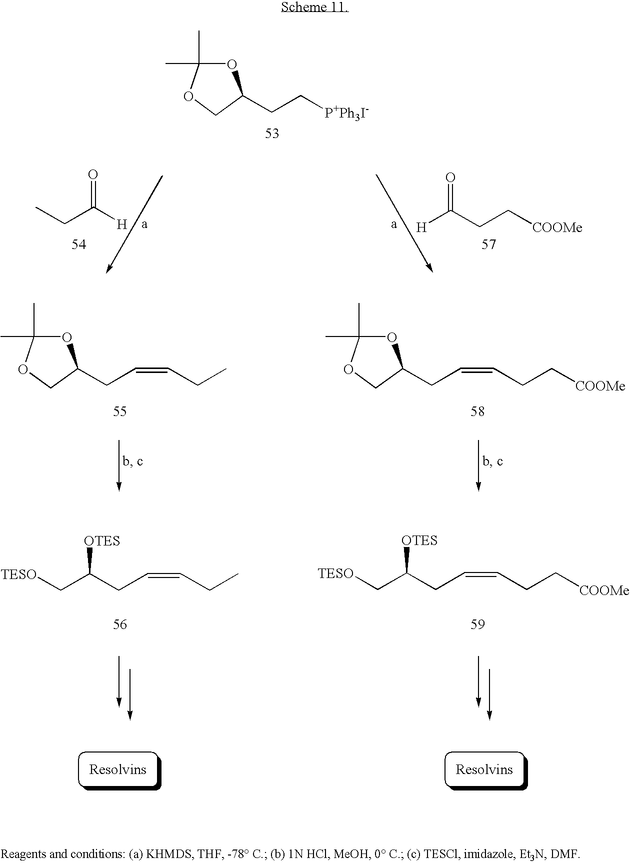

In conclusion, a concise total synthesis of 4(S), 17(S)-Resolvin D6 14, 4(R), 17(S)-Resolvin D6 15 and d-4-7(S),17(S)-Resolvin D5 33 has been achieved, making these novel lipid mediators available for further biological and pharmacological testing. An improved synthesis of key chiral intermediates based on a modification of Noyori's asymmetric transfer hydrogenation has been developed. A general approach towards Resolvins and other Eicosanoids based on malic acid has been developed.

In one aspect of the invention a process for the synthesis of the compound of structure 1 is disclosed:

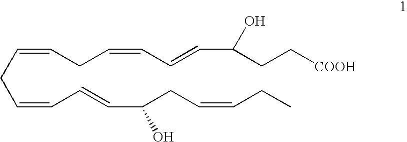

In one embodiment the process for the synthesis of the compound of structure 1 comprises the steps of:
a) converting the acid 2 to compound 3:

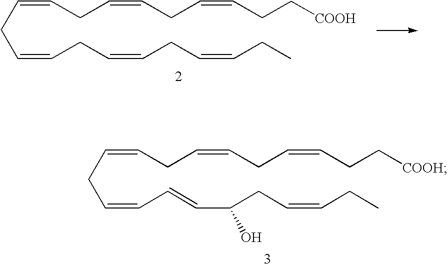

b) converting the compound 3 to compound 10:

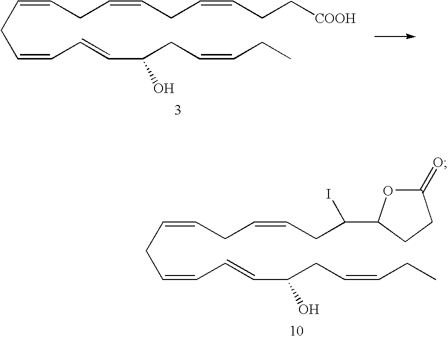

c) converting the compound 10 to compound 11:

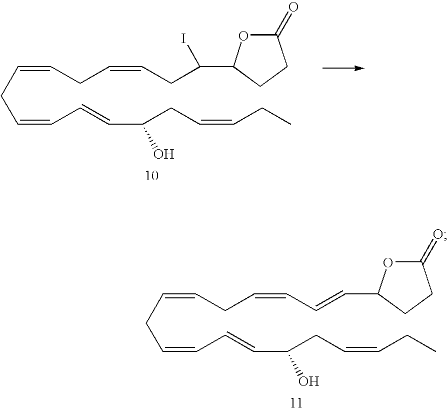

and
d) hydrolyzing compound 11 to the compound 1:

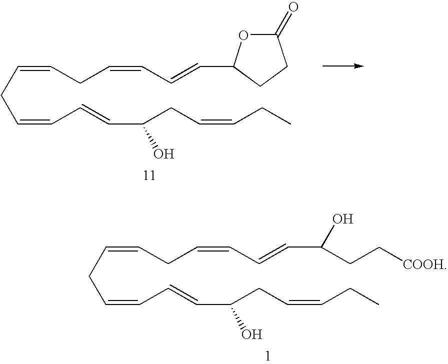

In another aspect of the invention a composition of a compound according to formulae 3, 10, or 11 is disclosed.

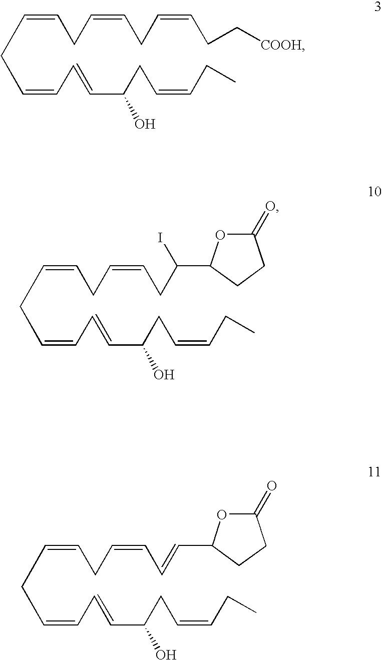

In one embodiment, with respect to the process for synthesis of compound 1, the “Step a” comprises:

lipoxygenase oxidation; and followed by

reduction of the intermediate hydroperoxide.

In one embodiment, with respect to the process for synthesis of compound 1, the lipoxygenase oxidation is carried out in the presence of a buffer.

In one embodiment, with respect to the process for synthesis of compound 1, the lipoxygenase oxidation is carried out in the presence of a 0.1 M borate buffer.

In one embodiment, with respect to the process for synthesis of compound 1, the lipoxygenase oxidation is carried out at between pH 8-10.

In one embodiment, with respect to the process for synthesis of compound 1, the lipoxygenase oxidation is carried out at between pH 9.

In one embodiment, with respect to the process for synthesis of compound 1, the reduction is carried out in the presence of a PPh₃.

In one embodiment, with respect to the process for synthesis of compound 1, the “Step b” comprises:

iodolactonization.

In one embodiment, with respect to the process for synthesis of compound 1, the iodolactonization is carried out in the presence of

I₂;

KI or NaI;

a base; and

a solvent.

In one embodiment, with respect to the process for synthesis of compound 1, the base is selected from NaHCO₃ and KHCO₃.

In one embodiment, with respect to the process for synthesis of compound 1, the solvent is selected from a 2:1 mixture of THF:water.

In one embodiment, with respect to the process for synthesis of compound 1, the “Step b” is carried out at −5 to +5° C.

In one embodiment, with respect to the process for synthesis of compound 1, the “Step c” comprises:

elimination.

In one embodiment, with respect to the process for synthesis of compound 1, the elimination is carried out in the presence of a base.

In one embodiment, with respect to the process for synthesis of compound 1, the base is DBU.

In one embodiment, with respect to the process for synthesis of compound 1, the “Step c” is carried out at 15-25° C.

In one embodiment, with respect to the process for synthesis of compound 1, the “Step d” comprises:

reaction with a base; and then

reaction with an acid.

In one embodiment, with respect to the “Step d” of the process for synthesis of compound 1, the base is selected from LiOH, KOH and NaOH.

In one embodiment, with respect to the “Step d” of the process for synthesis of compound 1, the base is LiOH.

In one embodiment, with respect to the “Step d” of the process for synthesis of compound 1, the reaction with a base is carried out in THF:H₂O mixture.

In one embodiment, with respect to the “Step d” of the process for synthesis of compound 1, the acid is selected from any acid which is able to generate pH 4.5.

In one embodiment, with respect to the “Step d” of the process for synthesis of compound 1, the acid is selected from any inorganic acid which is able to generate pH 4.5.

In one embodiment, with respect to the “Step d” of the process for synthesis of compound 1, the acid is selected from any phosphate buffer which is able to generate pH 4.5.

In one embodiment, with respect to the process for synthesis of compound 1, the “Step d” is carried out at 0-45° C. In another embodiment the “Step d” is carried out between 5-25° C.

In another embodiment the “Step d” is carried out between 20-25° C.

In another aspect of the invention a process for the synthesis of the compound of structure 14 is disclosed:

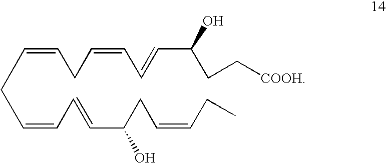

In one embodiment the process for the synthesis of the compound of structure 14 comprises the steps of:
a) converting the acid 2 to compound 3:

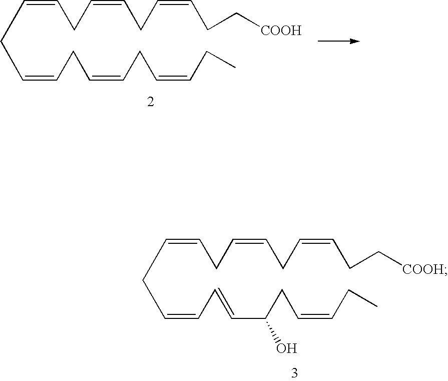

b) converting the compound 3 to compound 10:

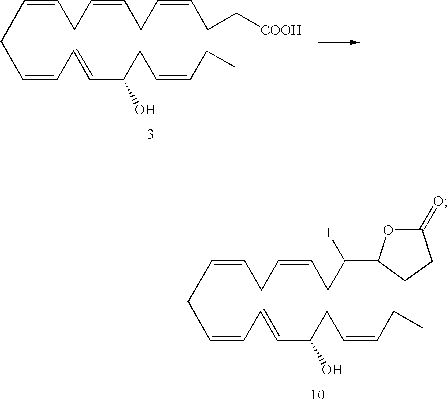

c) converting the compound 10 to compound 11:

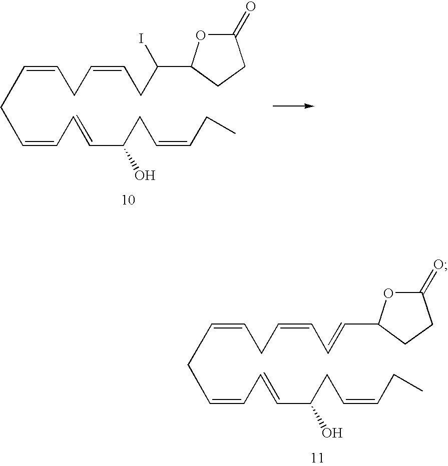

d) separating the isomers by chiral HPLC separation:

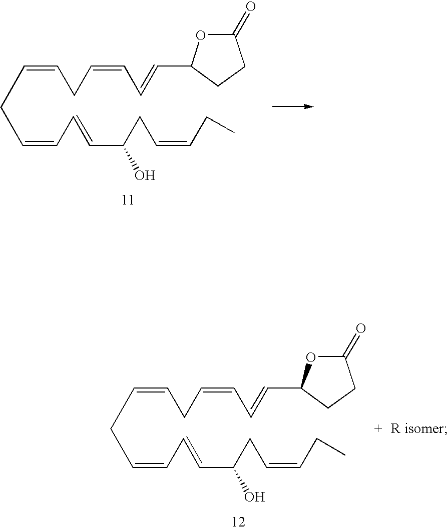

and
e) hydrolyzing the compound 12 to the compound 14:

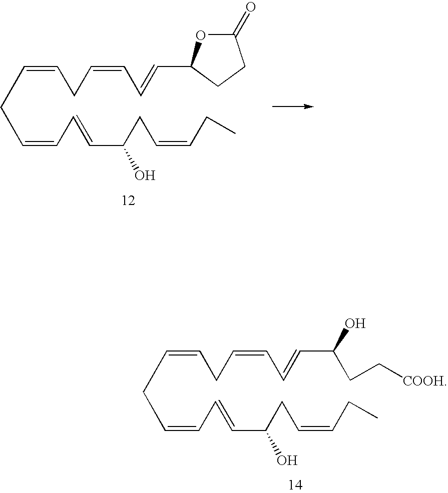

In yet another aspect of the invention, compounds according to formulae 12, or 14 are disclosed.

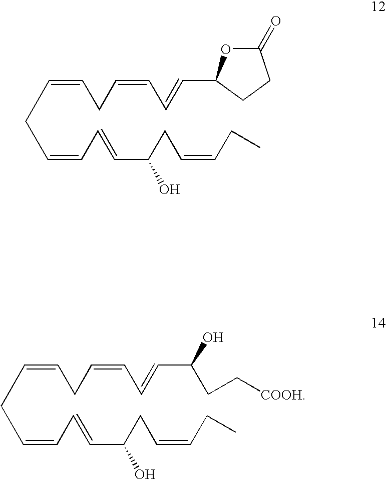

In another aspect of the invention, a process for the synthesis of the compound of structure 15 is disclosed:

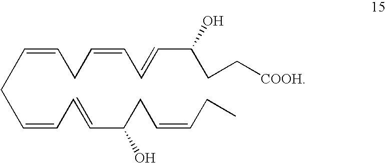

In one embodiment, the process for the synthesis of the compound of structure 15 comprises the steps of:
a) converting the acid 2 to compound 3:

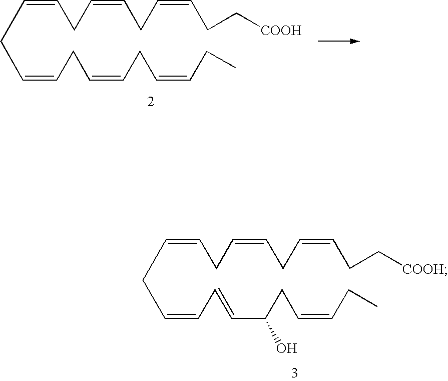

b) converting the compound 3 to compound 10:

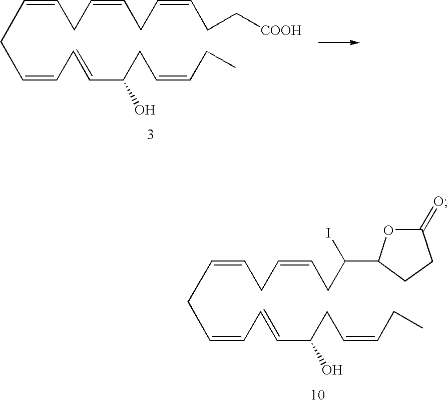

c) converting the compound 10 to compound 11:

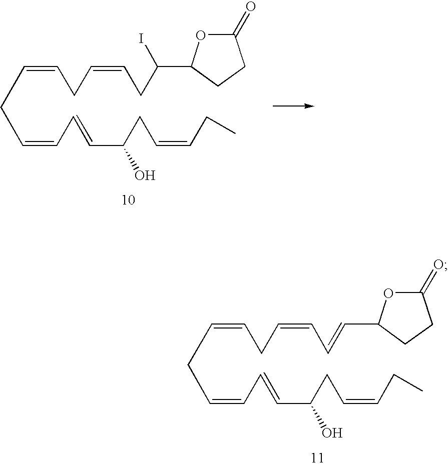

d) separating the isomers by chiral HPLC separation:

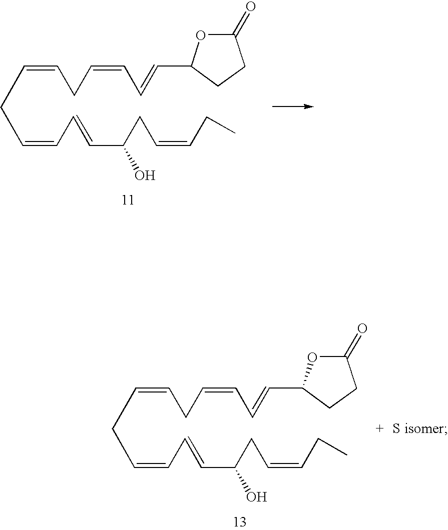

and
e) hydrolyzing the compound 13 to the compound 15:

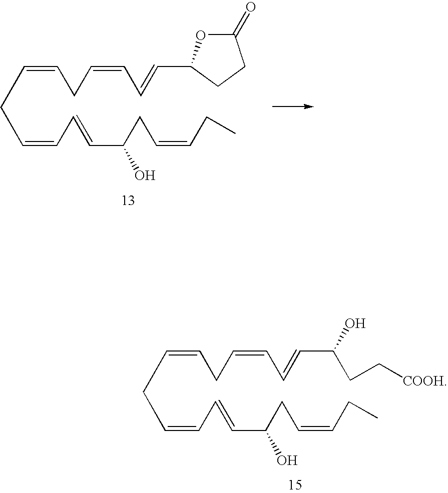

In yet another aspect of the invention compounds according to formulae 13 or 15 are disclosed:

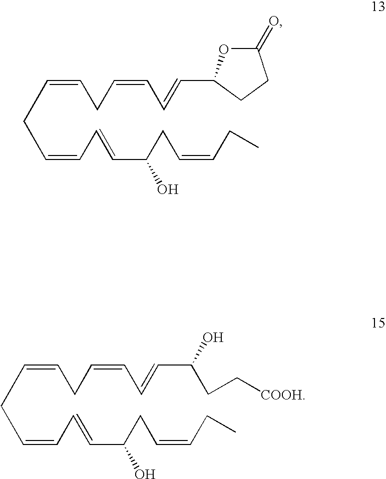

In one embodiment, with respect to the process for synthesis of compound 14 or 15, the “Step a” comprises:

lipoxygenase oxidation; and

reduction of the intermediate hydroperoxide.

In one embodiment, with respect to the process for synthesis of compound 14 or 15, the lipoxygenase oxidation is carried out in the presence of a buffer.

In one embodiment, with respect to the process for synthesis of compound 14 or 15, the lipoxygenase oxidation is carried out in the presence of a 0.1 M borate buffer.

In one embodiment, with respect to the process for synthesis of compound 14 or 15, the lipoxygenase oxidation is carried out at between pH 8-10.

In one embodiment, with respect to the process for synthesis of compound 14 or 15, the lipoxygenase oxidation is carried out at between pH 9.

In one embodiment, with respect to the process for synthesis of compound 14 or 15, the reduction is carried out in the presence of a PPh₃.

In one embodiment, with respect to the process for synthesis of compound 14 or 15, the “Step b” comprises:

iodolactonization.

In one embodiment, with respect to the process for synthesis of compound 14 or 15, the iodolactonization is carried out in the presence of

I₂;

KI or NaI;

a base; and

a solvent.

In one embodiment, with respect to the process for synthesis of compound 14 or 15, the base is selected from NaHCO₃ and KHCO₃.

In one embodiment, with respect to the process for synthesis of compound 14 or 15, the solvent is selected from a 2:1 mixture of THF:water.

In one embodiment, with respect to the process for synthesis of compound 14 or 15, the “Step b” is carried out at −5 to +5° C.

In one embodiment, with respect to the process for synthesis of compound 14 or 15, the “Step b” is carried out at 0° C.

In one embodiment, with respect to the process for synthesis of compound 14 or 15, the “Step c” comprises:

elimination.

In one embodiment, with respect to the process for synthesis of compound 14 or 15, the elimination is carried out in the presence of a base.

In one embodiment, with respect to the process for synthesis of compound 14 or 15, the base is DBU.

In one embodiment, with respect to the process for synthesis of compound 14 or 15, the “Step c” is carried out at 15-25° C.

In one embodiment, with respect to the “Step d” of the process for synthesis of compound 14 or 15, chiral HPLC separation is used for separating the isomers.

In one embodiment, with respect to the process for synthesis of compound 14 or 15, the “Step e” comprises:

reaction with a base; and then

reaction with an acid.

In one embodiment, with respect to the “Step e” of the process for synthesis of compound 14 or 15, the base is selected from LiOH, KOH and NaOH.

In one embodiment, with respect to the “Step e” of the process for synthesis of compound 14 or 15, the base is LiOH.

In one embodiment, with respect to the “Step e” of the process for synthesis of compound 14 or 15, the reaction with a base is carried out in THF:H₂O mixture.

In one embodiment, with respect to the “Step e” of the process for synthesis of compound 14 or 15, the acid is selected from any acid which is able to generate pH 4.5.

In one embodiment, with respect to the “Step e” of the process for synthesis of compound 14 or 15, the acid is selected from any inorganic acid which is able to generate pH 4.5.

In one embodiment, with respect to the “Step e” of the process for synthesis of compound 14 or 15, the acid is selected from any phosphate buffer which is able to generate pH 4.5.

In one embodiment, with respect to the process for synthesis of compound 14 or 15, the “Step e” is carried out at 0-45° C. In another embodiment the “Step e” is carried out between 5-25° C.

In another embodiment the “Step e” is carried out between 20-25° C.

In another aspect of the invention a process for the synthesis of the compound of structure 18 is disclosed:

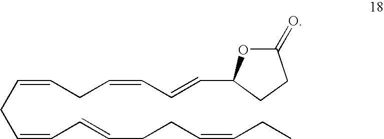

In one embodiment the process for the synthesis of the compound of structure 18 comprises the steps of:
a) converting the acid 2 to compound 16:

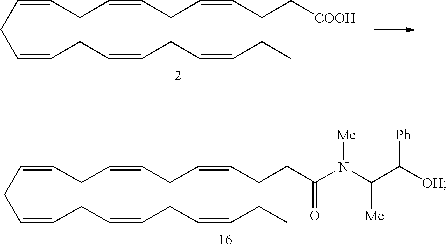

b) converting the compound 16 to compound 17:

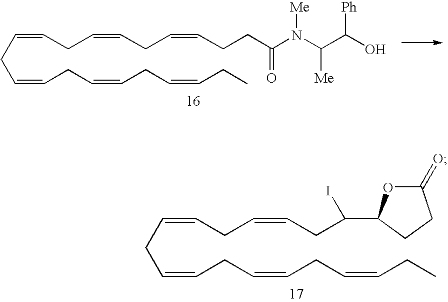

and
c) converting the compound 17 to compound 18:

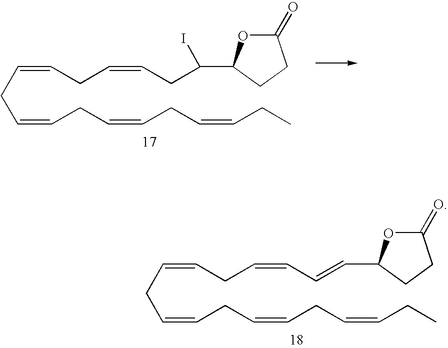

In one embodiment, with respect to the process for synthesis of compound 18, the “Step a” comprises:

formation of an acid chloride; and then

reacting the acid chloride with pseudoephedrine.

In one embodiment, with respect to “step a” of the process for synthesis of compound 18, the chlorination is performed using oxalyl chloride.

In one embodiment, with respect to “step a” of the process for synthesis of compound 18, the chlorination is performed in CH₂Cl₂.

In one embodiment, with respect to “step a” of the process for synthesis of compound 18, the chlorination is performed in the presence of DMF.

In one embodiment, with respect to “step a” of the process for synthesis of compound 18, the reaction of pseudoephedrine is performed in THF.

In one embodiment, with respect to “step a” of the process for synthesis of compound 18, the reaction of pseudoephedrine is performed in the presence of a base.

In one embodiment, with respect to “step a” of the process for synthesis of compound 18, the reaction of pseudoephedrine is performed in the presence of tert-amine.

In one embodiment, with respect to “step a” of the process for synthesis of compound 18, the reaction of pseudoephedrine is performed in the presence of triethylamine.

In one embodiment, with respect to “step b” of the process for synthesis of compound 18, the iodination is performed using I₂.

In one embodiment, with respect to “step b” of the process for synthesis of compound 18, the iodination is performed in THF:H₂O mixture.

In one embodiment, with respect to “step b” of the process for synthesis of compound 18, the elimination is performed in an inert solvent.

In one embodiment, with respect to “step b” of the process for synthesis of compound 18, the iodination is performed in benzene.

In one embodiment, with respect to “step b” of the process for synthesis of compound 18, the iodination is performed in the presence of a base.

In one embodiment, with respect to “step b” of the process for synthesis of compound 18, the iodination is performed in the presence of DBU.

In one embodiment, with respect to “step b” of the process for synthesis of compound 18, the iodination is performed at an ambient temperature.

In another aspect of the invention a process for the synthesis of the compound of structure 14 is disclosed:

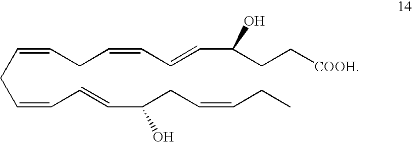

In one embodiment, the process for the synthesis of the compound of structure 14 comprises the steps of:
a) converting the acid 2 to compound 16:

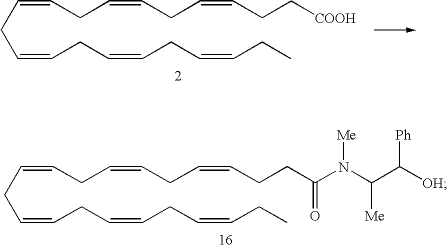

b) converting the compound 16 to compound 17:

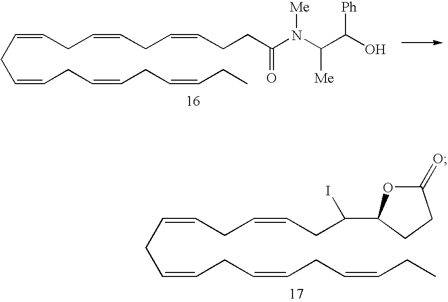

c) converting the compound 17 to compound 18:

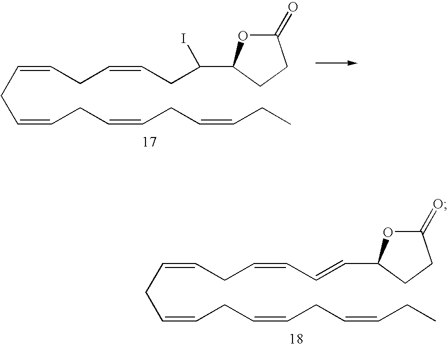

d) converting the compound 18 to compound 12:

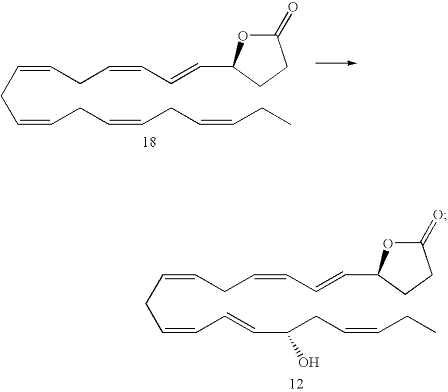

and
e) converting the compound 12 to compound 14:

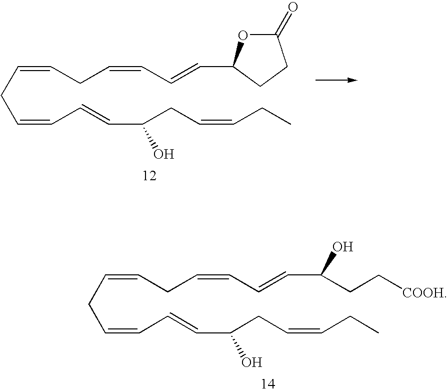

In one embodiment, with respect to the process for synthesis of compound 14, the “Step a” comprises:

formation of an acid chloride; and then

reacting the acid chloride with pseudoephedrine.

In one embodiment, with respect to “step a” of the process for synthesis of compound 14, the chlorination is performed using oxalyl chloride.

In one embodiment, with respect to “step a” of the process for synthesis of compound 14, the chlorination is performed in CH₂Cl₂.

In one embodiment, with respect to “step a” of the process for synthesis of compound 14, the chlorination is performed in the presence of DMF.

In one embodiment, with respect to “step a” of the process for synthesis of compound 14, the reaction of pseudoephedrine is performed in THF.

In one embodiment, with respect to “step a” of the process for synthesis of compound 14, the reaction of pseudoephedrine is performed in the presence of a base.

In one embodiment, with respect to “step a” of the process for synthesis of compound the reaction of pseudoephedrine is performed in the presence of tert-amine.

In one embodiment, with respect to “step a” of the process for synthesis of compound 14, the reaction of pseudoephedrine is performed in the presence of triethylamine.

In one embodiment, with respect to “step b” of the process for synthesis of compound 14, the iodination is performed using I₂.

In one embodiment, with respect to “step b” of the process for synthesis of compound 14, the iodination is performed in THF:H₂O mixture.

In one embodiment, with respect to “step b” of the process for synthesis of compound 14, the elimination is performed in an inert solvent.

In one embodiment, with respect to “step b” of the process for synthesis of compound 14, the iodination is performed in benzene.

In one embodiment, with respect to “step b” of the process for synthesis of compound 14, the iodination is performed in the presence of a base.

In one embodiment, with respect to “step b” of the process for synthesis of compound 14, the iodination is performed in the presence of DBU.

In one embodiment, with respect to “step b” of the process for synthesis of compound 14, the iodination is performed at an ambient temperature.

In one embodiment, with respect to the process for synthesis of compound 14, the “Step c” comprises:

elimination.

In one embodiment, with respect to the process for synthesis of compound 14, the elimination is carried out in the presence of a base.

In one embodiment, with respect to the process for synthesis of compound 14, the base is DBU.

In one embodiment, with respect to the process for synthesis of compound 14, the “Step c” is carried out at 15-25° C.

In one embodiment, with respect to the process for synthesis of compound 14, the “Step d” comprises:

lipoxygenase oxidation; and

reduction of the intermediate hydroperoxide.

In one embodiment, with respect to the process for synthesis of compound 14, the lipoxygenase oxidation is carried out in the presence of a buffer.

In one embodiment, with respect to the process for synthesis of compound 14, the lipoxygenase oxidation is carried out in the presence of a 0.1 M borate buffer.

In one embodiment, with respect to the process for synthesis of compound 14, the lipoxygenase oxidation is carried out at between pH 8-10.

In one embodiment, with respect to the process for synthesis of compound 14, the lipoxygenase oxidation is carried out at between pH 9.

In one embodiment, with respect to the process for synthesis of compound 14, the reduction is carried out in the presence of a PPh₃.

In one embodiment, with respect to the process for synthesis of compound 14, the “Step e” comprises:

reaction with a base; and then

reaction with an acid.

In one embodiment, with respect to the “Step e” of the process for synthesis of compound 14, the base is selected from LiOH, KOH and NaOH.

In one embodiment, with respect to the “Step e” of the process for synthesis of compound 14, the base is LiOH.

In one embodiment, with respect to the “Step e” of the process for synthesis of compound 14, the reaction with a base is carried out in THF:H₂O mixture.

In one embodiment, with respect to the “Step e” of the process for synthesis of compound 14, the acid is selected from any acid which is able to generate pH 4.5.

In one embodiment, with respect to the “Step e” of the process for synthesis of compound 14, the acid is selected from any inorganic acid which is able to generate pH 4.5.

In one embodiment, with respect to the “Step e” of the process for synthesis of compound 14, the acid is selected from any phosphate buffer which is able to generate pH 4.5.

In one embodiment, with respect to the process for synthesis of compound 14, the “Step e” is carried out at 0-45° C. In another embodiment the “Step e” is carried out between 5-25° C.

In another embodiment, the “Step e” is carried out between 20-25° C.

In another aspect of the invention a process for the synthesis of the compound of structure 24 is disclosed:

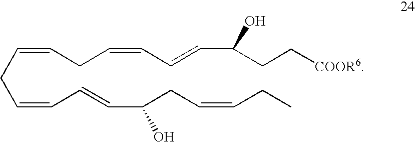

In one embodiment the process for the synthesis of the compound of structure 19 comprises the steps of:
a) converting the acid 2 to compound 19:

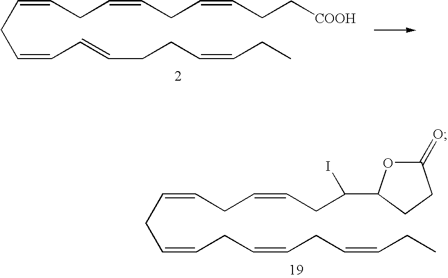

b) converting the compound 19 to compound 20:

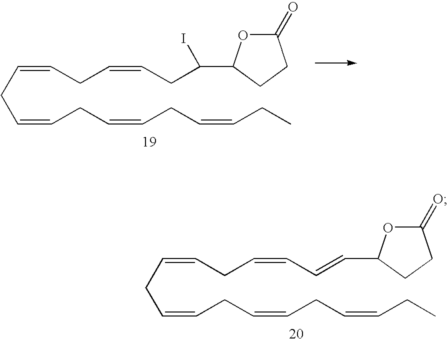

c) converting the compound 20 to compound 21:

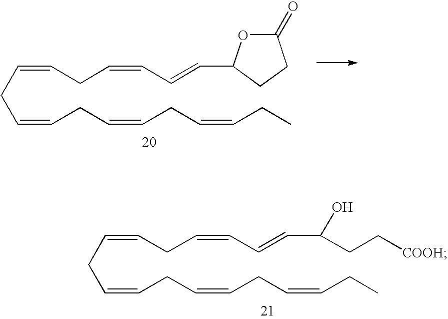

d) converting the compound 21 to compound 22:

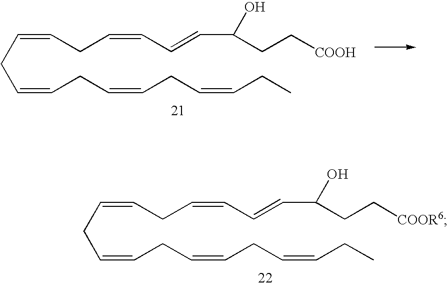

e) converting the compound 22 to compound 23:

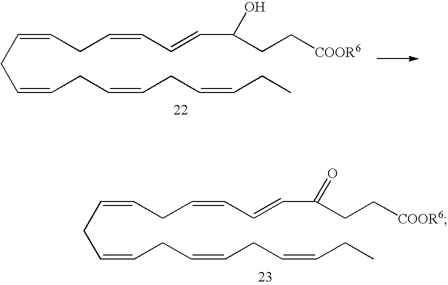

and
f) reducing the compound 23 to the compound 24:

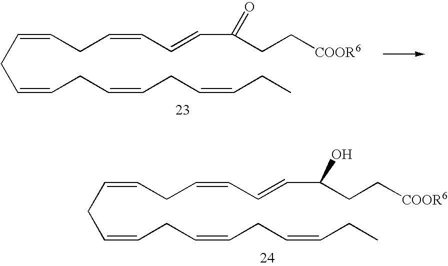

wherein R⁶ is alkyl or benzyl.

In one embodiment, with respect to the process for synthesis of compound 24, R⁶ is Me, Et, n-Bu, benzyl or t-Bu.

In one embodiment, with respect to the process for synthesis of compound 24, R⁶ is Me.

In one embodiment, with respect to the process for synthesis of compound 24, the “Step a” comprises:

iodolactonization.

In one embodiment, with respect to the process for synthesis of compound 24, the iodolactonization is carried out in the presence of

I₂;

KI or NaI;

a base; and

a solvent.

In one embodiment, with respect to the process for synthesis of compound 24, the base is selected from NaHCO₃ and KHCO₃.

In one embodiment, with respect to the process for synthesis of compound 24, the solvent is selected from a 2:1 mixture of THF:water.

In one embodiment, with respect to the process for synthesis of compound 24, the “Step a” is carried out at −5 to +5° C.

In one embodiment, with respect to the process for synthesis of compound 24, the “Step a” is carried out at 0° C.

In one embodiment, with respect to the process for synthesis of compound 24, the “Step b” is performed in an inert solvent.

In one embodiment, with respect to the process for synthesis of compound 24, the “Step b” is performed in benzene.

In one embodiment, with respect to the process for synthesis of compound 24, the “Step b” is performed in presence of a base.

In one embodiment, with respect to the process for synthesis of compound 24, the “Step b” is performed in presence of DBU.

In one embodiment, with respect to the process for synthesis of compound 24, the “Step b” is performed at an ambient temperature.

In one embodiment, with respect to the process for synthesis of compound 24, the “Step e” comprises:

reaction with a base; and then

reaction with an acid.

In one embodiment, with respect to the “Step e” of the process for synthesis of compound 24, the base is selected from LiOH, KOH and NaOH.

In one embodiment, with respect to the “Step e” of the process for synthesis of compound 24, the base is LiOH.

In one embodiment, with respect to the “Step e” of the process for synthesis of compound 24, the reaction with a base is carried out in THF:H₂O mixture.

In one embodiment, with respect to the “Step e” of the process for synthesis of compound 24, the acid is selected from any acid which is able to generate pH 4.5.

In one embodiment, with respect to the “Step e” of the process for synthesis of compound 24, the acid is selected from any inorganic acid which is able to generate pH 4.5.

In one embodiment, with respect to the “Step e” of the process for synthesis of compound 24, the acid is selected from any phosphate buffer which is able to generate pH 4.5.

In one embodiment, with respect to the process for synthesis of compound 24, the “Step e” is carried out at 0-45° C. In another embodiment the “Step e” is carried out between 5-25° C.

In another embodiment the “Step e” is carried out between 20-25° C.

In one embodiment, with respect to the process for synthesis of compound 24, the “Step d” is performed using diazomethane.

In one embodiment, with respect to the process for synthesis of compound 24, the “Step d” is performed in an inert solvent.

In one embodiment, with respect to the process for synthesis of compound 24, the “Step d” is performed in diethyl ether.

In one embodiment, with respect to the process for synthesis of compound 24, the “Step e” is performed using any conventional oxidizing agent.

In one embodiment, with respect to the process for synthesis of compound 24, the “Step e” is performed using Dess-Martin periodinane reagent.

In one embodiment, with respect to the process for synthesis of compound 24, the “Step e” is performed in CH₂Cl₂.

In one embodiment, with respect to the process for synthesis of compound 24, the “Step f” involves an asymmetric reduction.

In one embodiment, with respect to the process for synthesis of compound 24, the “Step f” is performed using “Corey-chiral-reduction”. In one embodiment, the reduction is carried out using (R)-2-methyl-CBS-oxazaborolidine and BH₃.Me₂S. In one embodiment, the reduction is carried out in toluene.

In one embodiment, with respect to the process for synthesis of compound 24, the “Step f” is performed using “Corey-chiral-reduction”. In one embodiment, the reduction is carried out using (R)-2-methyl-CBS-oxazaborolidine and catecholborane. In one embodiment, the reduction is carried out in CH₂Cl₂.

In one embodiment, with respect to the process for synthesis of compound 24, the “Step f” is performed using “Transfer hydrogenation”. In one embodiment, the reduction is carried out using Ru-TsDPEN. In one embodiment, the reduction is carried out in i-PrOH.

In one embodiment, with respect to the process for synthesis of compound 24, the “Step f” is performed using “Noyori-chiral-reduction”. In one embodiment, the reduction is carried out using BINAL-H. In one embodiment, the reduction is carried out in THF.

In another aspect of the invention a process for the synthesis of the compound of structure 33 is disclosed:

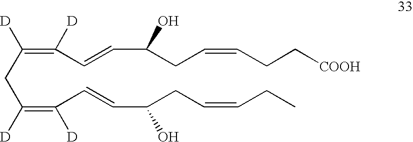

In one embodiment the process for the synthesis of the compound of structure 33 comprises the steps of:
a) condensing compound 25 with compound 26 to afford compound 27:

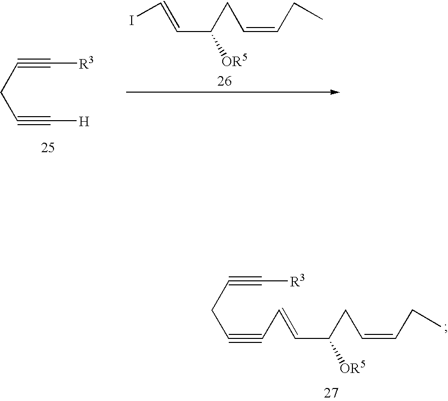

b) converting the compound 27 to compound 28:

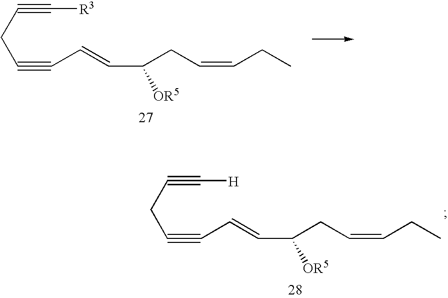

c) coupling of the compound 28 with compound 29 to give compound 30:

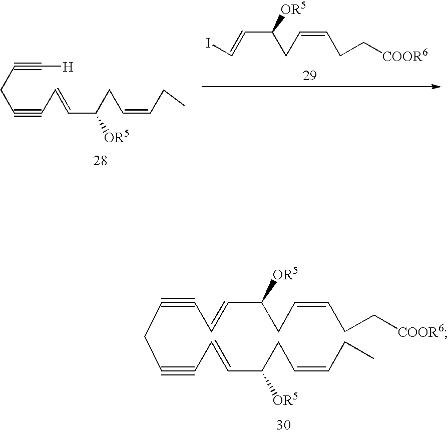

d) deprotecting compound 30 to the compound 31:

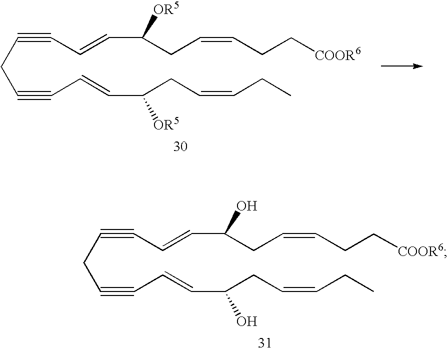

e) converting compound 31 to the compound 32:

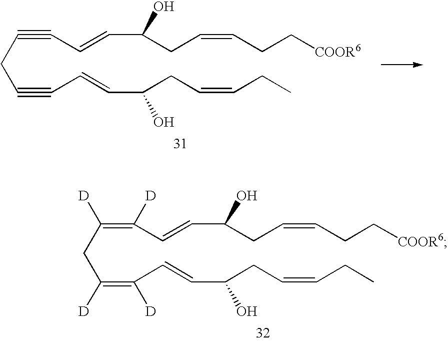

and
f) converting compound 32 to the compound 33:

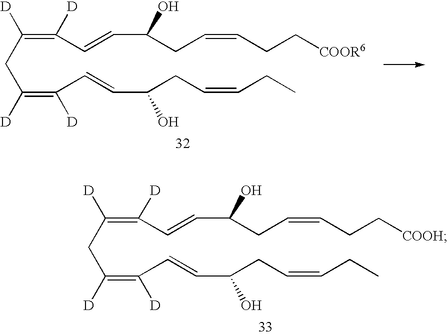

wherein each R³ is silyl protecting group; and R⁵ is silyl or O-protecting group; and R⁶ is alkyl or benzyl.

In one embodiment, with respect to the process for synthesis of compound 33, R³ is TBS, TES, TMS, TBDMS, or TIPS.

In one embodiment, with respect to the process for synthesis of compound 33, R³ is TMS.

In one embodiment, with respect to the process for synthesis of compound 33, R⁵ is TBS, TES, TMS, TBDMS, or TIPS.

In one embodiment, with respect to the process for synthesis of compound 33, R⁶ is Me or Et.

In one embodiment, with respect to the process for synthesis of compound 33, R⁶ is Me.

In one embodiment, with respect to the process for synthesis of compound 33, the “Step e” is carried out using Zn(Cu/Ag). In another embodiment the “Step e” is carried out in CD₃OD:D₂O mixture. In another embodiment the “Step e” is carried out in 1:1 CD₃OD:D₂O mixture. In another embodiment the “Step e” is carried out at 0-50° C. In another embodiment the “Step e” is carried out at 20-50° C. In another embodiment the “Step e” is carried out at 40° C.

In one embodiment, with respect to the process for synthesis of compound 33, the “Step f” comprises:

reaction with a base; and then

reaction with an acid.

In one embodiment, with respect to the “Step f” of the process for synthesis of compound 33, the base is selected from LiOH, KOH and NaOH.

In one embodiment, with respect to the “Step f” of the process for synthesis of compound 33, the base is LiOH.

In one embodiment, with respect to the “Step f” of the process for synthesis of compound 33, the reaction with a base is carried out in THF.

In one embodiment, with respect to the “Step f” of the process for synthesis of compound 33, the reaction with a base is carried out in THF:H₂O mixture.

In one embodiment, with respect to the “Step f” of the process for synthesis of compound 33, the acid is selected from any acid which is able to generate pH 4.5.

In one embodiment, with respect to the “Step f” of the process for synthesis of compound 33, the acid is selected from any inorganic acid which is able to generate pH 4.5.

In one embodiment, with respect to the “Step f” of the process for synthesis of compound 33, the acid is selected from any phosphate buffer which is able to generate pH 4.5.

In one embodiment, with respect to the “Step f” of the process for synthesis of compound 33, the acid is NaH₂PO₄.

In one embodiment, with respect to the “Step f” of the process for synthesis of compound 33, the acid reaction is carried out in the presence of EtOAc.

In another aspect of the invention a process for the synthesis of the compound of structure 36 is disclosed:

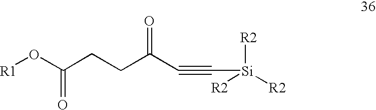

wherein each R¹ and R² are independently selected from alkyl.

In one embodiment the process for the synthesis of the compound of structure 36 comprises the step of:
a) acylating compound 34 with compound 35 in presence of a catalytic amount of InBr₃ to afford compound 36:

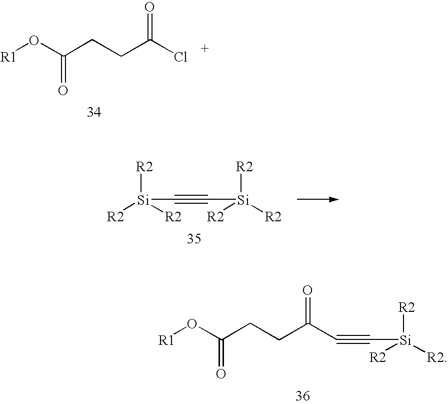

In another aspect of the invention a process for the synthesis of the compound of structure 38 is disclosed:

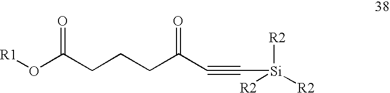

wherein each R¹ and R² are independently selected from alkyl.

In one embodiment, the process for the synthesis of the compound of structure 38 comprises the step of:
a) acylating compound 37 with compound 35 in presence of a catalytic amount of InBr₃ to afford compound 38:

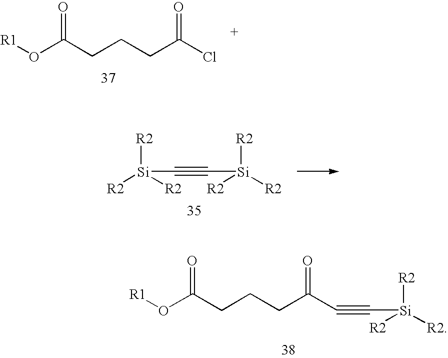

In another aspect of the invention, a process for the synthesis of the compound of structure 40 is disclosed:

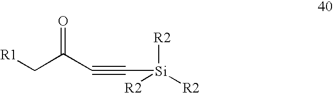

wherein each R² is independently selected from alkyl.

In one embodiment, the process for the synthesis of the compound of structure 40 comprises the step of:
a) acylating compound 39 with compound 35 in presence of a catalytic amount of InBr₃ to afford compound 38:

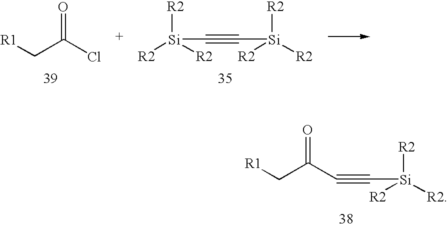

In one embodiment, with respect to the process for synthesis of compound 36, 38 or 40, R¹ is Me.

In one embodiment, with respect to the process for synthesis of compound 36, 38 or 40, R² is Me.

In one embodiment, with respect to the process for synthesis of compound 36, 38 or 40, the catalytic amount of InBr₃ is 2-10% of the acid chloride by weight.

In one embodiment, with respect to the process for synthesis of compound 36, 38 or 40, the catalytic amount of InBr₃ is 5% of the acid chloride by weight.

In one embodiment, with respect to the process for synthesis of compound 36, 38 or 40, the step a is carried out in an inert solvent.

In one embodiment, with respect to the process for synthesis of compound 36, 38 or 40, step a is carried out in CH₂Cl₂.

In another aspect of the invention a process for the synthesis of the compound of structure 45 is disclosed:

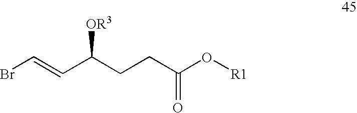

wherein each R¹ is alkyl; and R³ is a protecting group.

In one embodiment the process for the synthesis of the compound of structure 45 comprises the steps of:
a) reducing the compound 36 to compound 41:

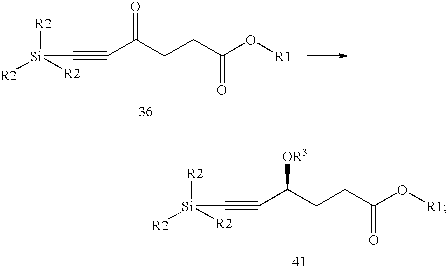

b) protecting the alcohol 41 to compound 42:

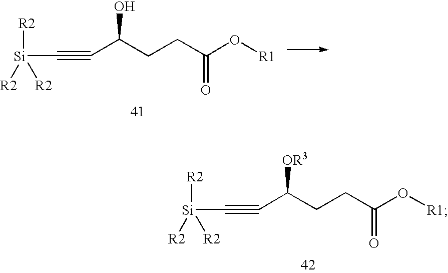

c) deprotecting the compound 42 to compound 43:

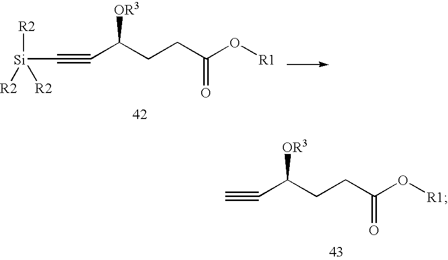

d) converting the compound 43 to compound 44:

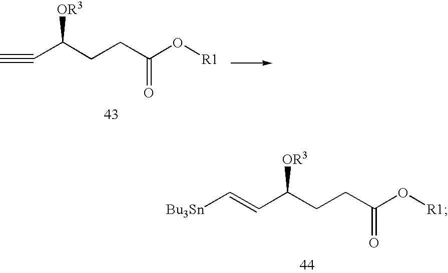

and
e) converting the compound 44 to compound 45:

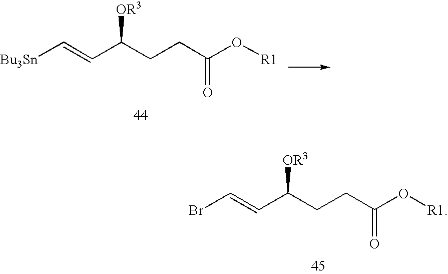

In another aspect of the invention a process for the synthesis of the compound of structure 46 is disclosed:

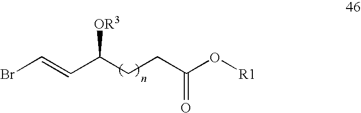

wherein each R¹ is alkyl; R³ is a protecting group; and n is 2, 3, 4 or 5.

In one embodiment the process for the synthesis of the compound of structure 46 comprises the steps of
a) reducing the compound 38 to compound 38B:

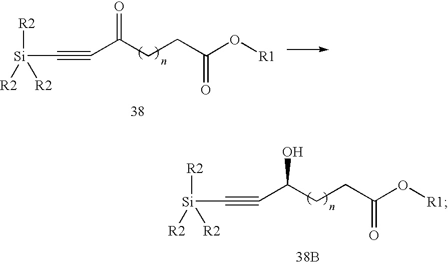

b) protecting the alcohol 38B to compound 38C:

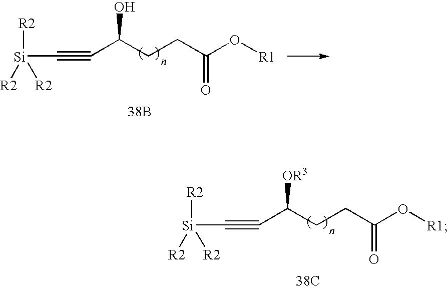

c) deprotecting the compound 38C to compound 38D:

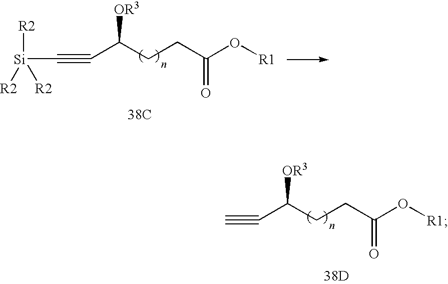

d) converting the compound 38D to compound 38E:

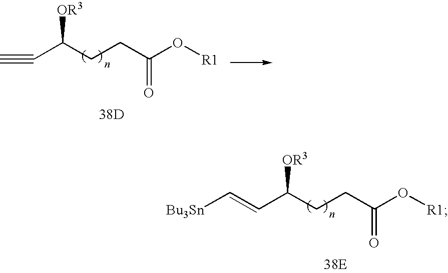

and
e) converting the compound 38E to compound 46:

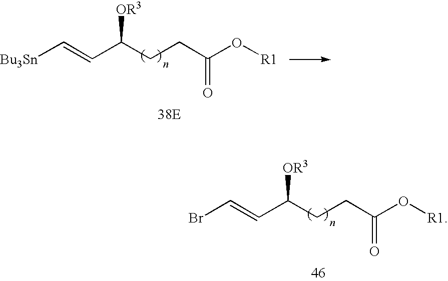

In one embodiment, the process for the synthesis of the compound of structure 46, n is 2, 3, 4 or 5.

In one embodiment, the process for the synthesis of the compound of structure 46, n is 2.

In another aspect of the invention, a process for the synthesis of the compound of structure 47 is disclosed:

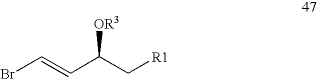

wherein each R¹ is alkyl; R³ is a protecting group.

In one embodiment, the process for the synthesis of the compound of structure 47 comprises the steps of:
a) reducing the compound 40 to compound 40B:

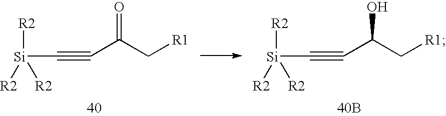

b) protecting the alcohol 40B to compound 40C:

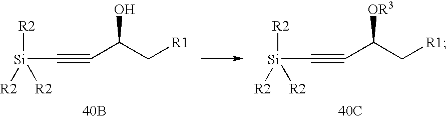

c) deprotecting the compound 40C to compound 40D:

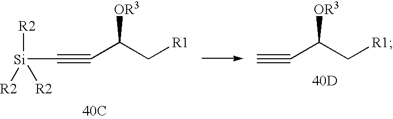

d) converting the compound 40D to compound 40E:

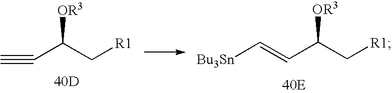

and
e) converting the compound 40E to compound 47:

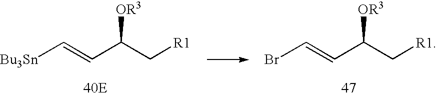

In one embodiment, with respect to the process for synthesis of compound 45, 46 or 47, R¹ is Me.

In one embodiment, with respect to the process for synthesis of compound 45, 46 or 47, R³ is any protecting group.

In one embodiment, with respect to the process for synthesis of compound 45, 46 or 47, R³ is an ester group.

In one embodiment, with respect to the process for synthesis of compound 45, 46 or 47, R³ is a silyl protecting group.

In one embodiment, with respect to the process for synthesis of compound 45, 46 or 47, R³ is TBS, TES, TMS, TBDMS, or TIPS.

In one embodiment, with respect to the process for synthesis of compound 45, 46 or 47, R³ is TBS.

In one embodiment, with respect to the process for synthesis of compound 45, 46 or 47, R³ is TIPS, or TBDMS.

In one embodiment, with respect to the process for synthesis of compound 45, 46 or 47, R³ is any O-protecting group.

In one embodiment, with respect to the process for synthesis of compound 45, 46 or 47, R³ is benzyl, THP or any alkyl ester group.

In one embodiment, with respect to the process for synthesis of compound 45, 46 or 47, the “Step a” is performed using any chiral reducing agent.

In one embodiment, with respect to the process for synthesis of compound 45, 46 or 47, the “Step a” is performed using [(S,S)-TsDPEN)Ru(p-cymene]Cl₂ reagent. In one embodiment, the reaction is carried out in an alcohol. In one embodiment, the reaction is carried out in i-PrOH.

In one embodiment, with respect to the process for synthesis of compound 45, 46 or 47, the “Step b” is performed using any silyl chloride reagent. In one embodiment, the reaction is performed using TBSCl, TESCl, TBDMSCl, or TIPSCl. In one embodiment, the reaction is carried out in the presence of imidazole. In one embodiment, the reaction is carried out in DMF.

In one embodiment, with respect to the process for synthesis of compound 45, 46 or 47, the “Step c” is performed using any conventional reagent for selective desilation of silyl-ethynyl group. In one embodiment, the reaction is performed using AgNO₃ and NaCN. In one embodiment, the reaction is carried out in MeOH. In one embodiment, the reaction is carried out in MeOH and H₂O.

In one embodiment, with respect to the process for synthesis of compound 45, 46 or 47, the “Step d” is performed using (Bu)₃SnH. In one embodiment, the reaction is carried out in the presence of AIBN. In one embodiment, the reaction is carried out at 100-150° C. In one embodiment, the reaction is carried out at 130° C.

In one embodiment, with respect to the process for synthesis of compound 45, 46 or 47, the “Step e” is performed using Br₂. In one embodiment, the reaction is carried out in an inert solvent. In one embodiment, the reaction is carried out in CH₂Cl₂. In one embodiment, the reaction is carried out at −10 to +10° C. In one embodiment, the reaction is carried out at −5° C.

In another aspect of the invention a process for the synthesis of the compound of structure 53 is disclosed:

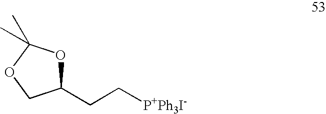

In one embodiment, the process for the synthesis of the compound of structure 53 comprises the steps of:
a) reducing the compound 48 to compound 49:

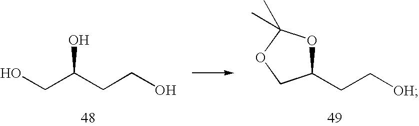

b) protecting the alcohol 49 to compound 51:

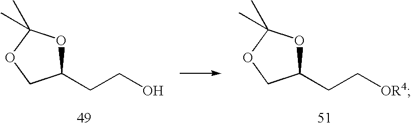

c) deprotecting the compound 51 to compound 52:

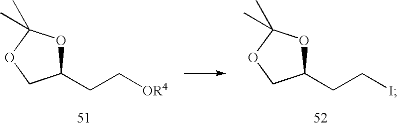

and
d) converting the compound 52 to compound 53:

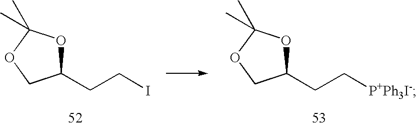

wherein R⁴ is a protecting group.

In one embodiment, with respect to the process for synthesis of compound 53, R⁴ is a tosylate, mesylate, or besylate.

In one embodiment, with respect to the process for synthesis of compound 53, R⁴ is a tosylate.

In one embodiment, with respect to the process for synthesis of compound 53, the “Step a” is performed using acetone. In one embodiment, the reaction is carried out in the presence of TsOH.

In one embodiment, with respect to the process for synthesis of compound 53, the “Step b” is performed using TsCl. In one embodiment, the reaction is carried out in the presence of Et₃N. In one embodiment, the reaction is carried out in CH₂Cl₂.

In one embodiment, with respect to the process for synthesis of compound 453, the “Step c” is performed using NaI. In one embodiment, the reaction is carried out in the presence of Cu. In one embodiment, the reaction is carried out in acetone.

In one embodiment, with respect to the process for synthesis of compound 53, the “Step d” is performed using PPh₃. In one embodiment, the reaction is carried out in the presence of NaHCO₃. In one embodiment, the reaction is carried out in MeCN.

In another aspect of the invention a process for the synthesis of the compound of structure 56 is disclosed:

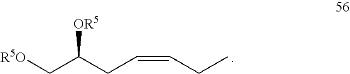

In one embodiment, the process for the synthesis of the compound of structure 56 comprises the steps of:
a) converting the compound 53 to compound 55:

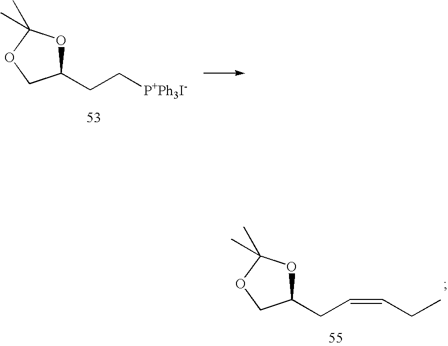

b) converting the compound 55 to compound 55B:

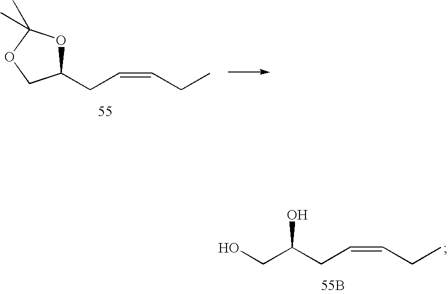

and
c) protecting the compound 55B to compound 56:

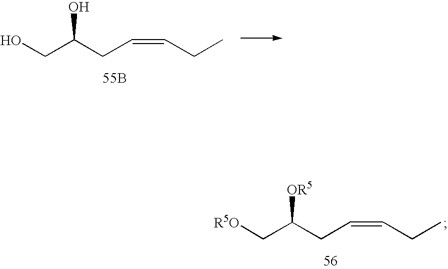

wherein R⁵ is a protecting group.

In another aspect of the invention a process for the synthesis of the compound of structure 59 is disclosed:

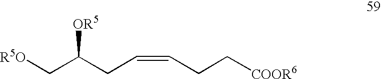

In one embodiment, the process for the synthesis of the compound of structure 59 comprises the steps of:
a) converting the compound 53 to compound 58:

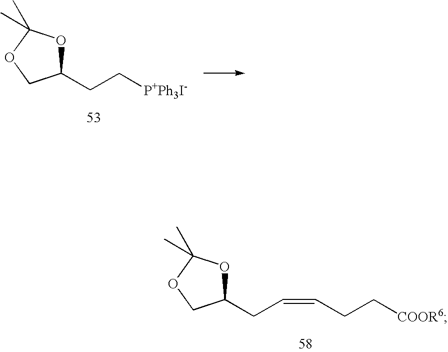

b) converting the compound 58 to compound 58B:

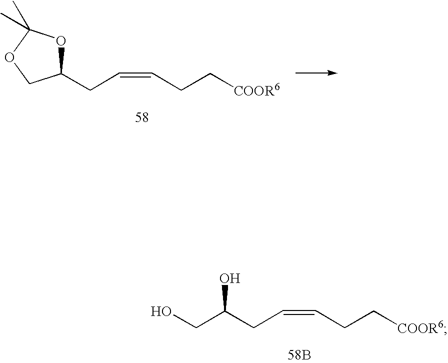

and
c) protecting the compound 55B to compound 56:

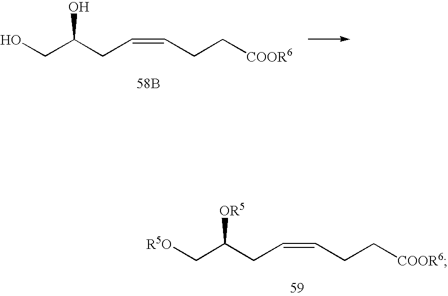

wherein R⁵ is a protecting group; and R⁶ is alkyl or benzyl.

In one embodiment, with respect to the process for synthesis of compound 56 or 59, R⁶ is Me or Et.

In one embodiment, with respect to the process for synthesis of compound 56 or 59, R⁵ is any protecting group.

In one embodiment, with respect to the process for synthesis of compound 56 or 59, R⁵ is an ester group.

In one embodiment, with respect to the process for synthesis of compound 56 or 59, R⁵ is a silyl protecting group.

In one embodiment, with respect to the process for synthesis of compound 56 or 59, R⁵ is TBS, TES, TMS, TBDMS, or TIPS.

In one embodiment, with respect to the process for synthesis of compound 56 or 59, the “Step a” is performed using KHMDS. In one embodiment, the reaction is carried out in the THF. In one embodiment, the reaction is carried out at −78° C.

In one embodiment, with respect to the process for synthesis of compound 56 or 59, the “Step b” is performed using 1N HCl. In one embodiment, the reaction is carried out in the MeOH. In one embodiment, the reaction is carried out at −10 to +10° C. In one embodiment, the reaction is carried out at 0° C.

In one embodiment, with respect to the process for synthesis of compound 56 or 59, the “Step c” is performed using any silylating agent. In one embodiment, the reaction is carried out using any silyl chloride reagent. In one embodiment, the reaction is carried out using TMSCl, TESCl, TBDMSCl or TIPSCl. In one embodiment, the reaction is carried out using TESCl. In one embodiment, the reaction is carried out in the presence of imidazole. In one embodiment, the reaction is carried out in the presence of Et₃N. In one embodiment, the reaction is carried out in the presence of DMF.

In another aspect of the invention a process for the synthesis of the compound of structure 8 is disclosed:

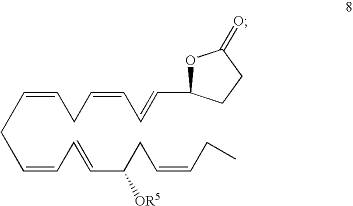

wherein R⁵ is a O-protecting group;

In one embodiment the process for the synthesis of the compound of structure 56 comprises the steps of:
a) converting the acid 2 to compound 3:

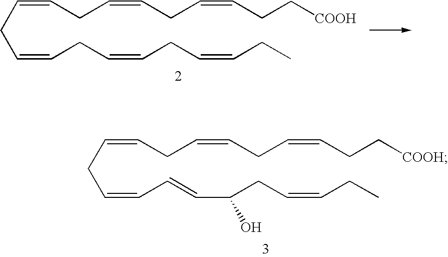

b) converting the compound 3 to compound 4:

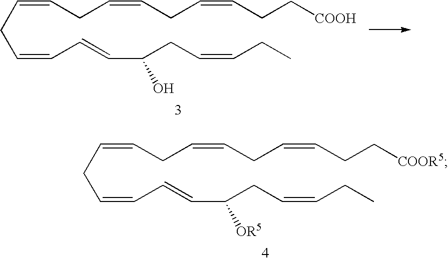

c) converting the compound 4 to compound 5:

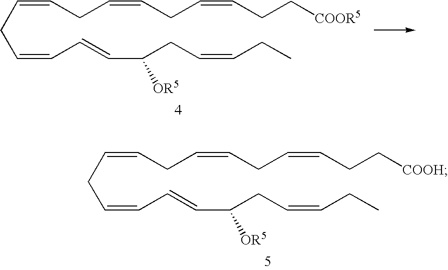

d) converting the compound 5 to compound 6:

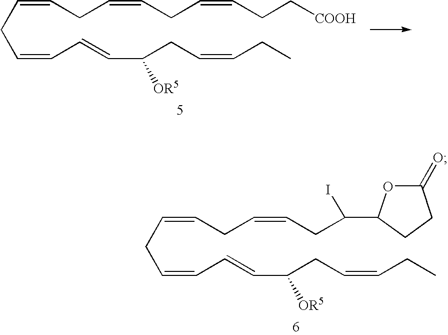

e) converting the compound 6 to compound 7:

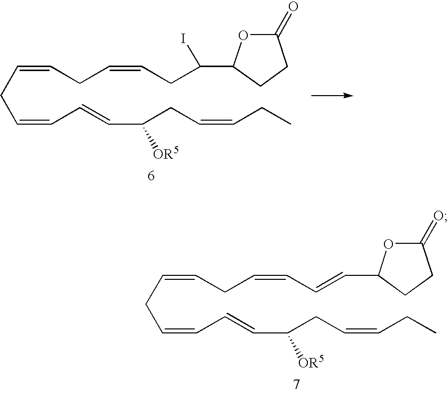

and
f) converting compound 7 to the compound 8:

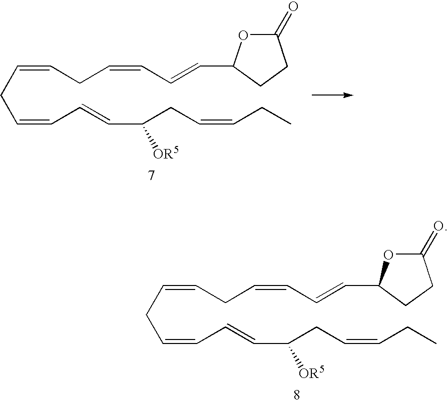

In another aspect of the invention a process for the synthesis of the compound of structure 9 is disclosed:

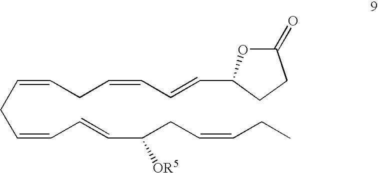

wherein R⁵ is a O-protecting group.

In one embodiment the process for the synthesis of the compound of structure 56 comprises the steps of
a) converting the acid 2 to compound 3:

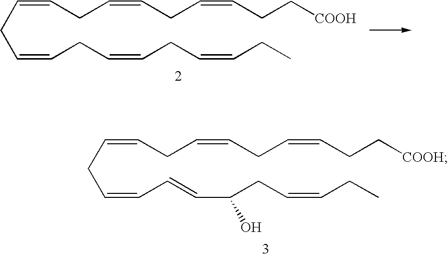

b) converting the compound 3 to compound 4:

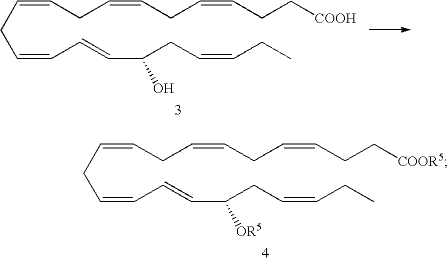

c) converting the compound 4 to compound 5:

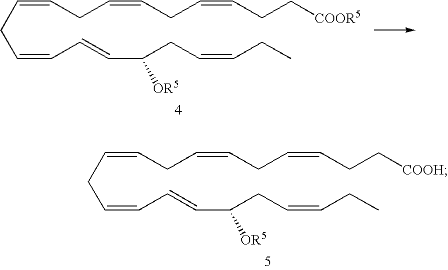

d) converting the compound 5 to compound 6:

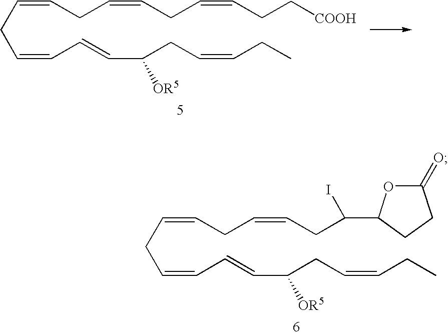

e) converting the compound 6 to compound 7:

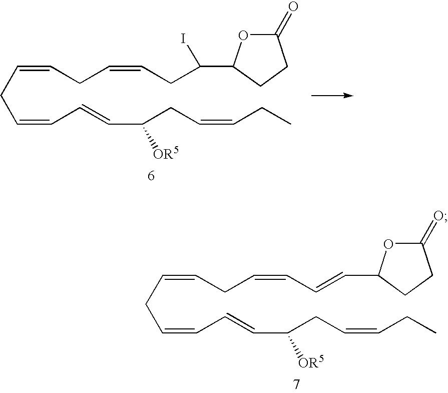

and
f) converting compound 7 to the compound 8:

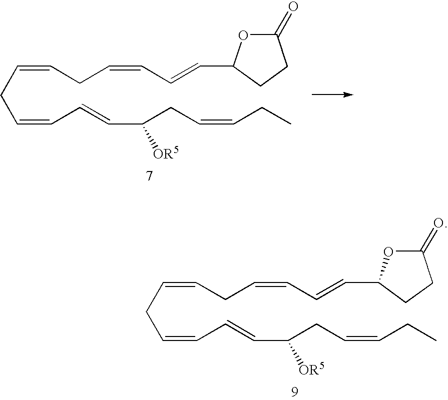

In one embodiment, with respect to the process according to process to synthesis of compound 8 or 9, R⁵ is a silyl protecting group.

In one embodiment, with respect to the process according to process to synthesis of compound 8 or 9, R⁵ is a TBS, TES, TMS, TBDMS, or TIPS.

In one embodiment, with respect to the process for synthesis of compound 8 or 9, the “Step a” comprises:

lipoxygenase oxidation; and followed by

reduction of the intermediate hydroperoxide.

In one embodiment, with respect to the process for synthesis of compound 8 or 9, the lipoxygenase oxidation is carried out in the presence of a buffer.

In one embodiment, with respect to the process for synthesis of compound 8 or 9, the lipoxygenase oxidation is carried out in the presence of a 0.1 M borate buffer.

In one embodiment, with respect to the process for synthesis of compound 8 or 9, the lipoxygenase oxidation is carried out at between pH 8-10.

In one embodiment, with respect to the process for synthesis of compound 8 or 9, the lipoxygenase oxidation is carried out at between pH 9.

In one embodiment, with respect to the process for synthesis of compound 8 or 9, the reduction is carried out in the presence of a PPh₃.

In one embodiment, with respect to the process for synthesis of compound 8 or 9, the “Step b” is performed using any silylating agent. In one embodiment, the reaction is carried out using any silyl chloride reagent. In one embodiment, the reaction is carried out using TMSCl, TESCl, TBDMSCl or TIPSCl. In one embodiment, the reaction is carried out using TESCl. In one embodiment, the reaction is carried out in the presence of imidazole. In one embodiment, the reaction is carried out in the presence of Et₃N. In one embodiment, the reaction is carried out in the presence of DMF.

In one embodiment, with respect to the process for synthesis of compound 8 or 9, the “Step c” is performed using K₂CO₃. In one embodiment, the reaction is carried out in the presence of THF, H₂O and MeOH. In one embodiment, the reaction is carried out at an ambient temperature.

In one embodiment, with respect to the process for synthesis of compound 8 or 9, the “Step d” comprises:

iodolactonization.

In one embodiment, with respect to the process for synthesis of compound 8 or 9, the iodolactonization is carried out in the presence of

I₂;

KI or NaI;

a base; and

a solvent.

In one embodiment, with respect to the process for synthesis of compound 8 or 9, the base is selected from NaHCO₃ and KHCO₃.

In one embodiment, with respect to the process for synthesis of compound 8 or 9, the solvent is selected from a 2:1 mixture of THF:water.

In one embodiment, with respect to the process for synthesis of compound 8 or 9, the “Step d” is carried out at −5 to +5° C.

In one embodiment, with respect to the process for synthesis of compound 8 or 9, the “Step d” is carried out at 0° C.

In one embodiment, with respect to the process for synthesis of compound 8 or 9, the “Step e” is performed in an inert solvent.

In one embodiment, with respect to the process for synthesis of compound 8 or 9, the “Step e” is performed in benzene.

In one embodiment, with respect to the process for synthesis of compound 8 or 9, the “Step e” is performed in presence of a base.

In one embodiment, with respect to the process for synthesis of compound 8 or 9, the “Step e” is performed in presence of DBU.

In one embodiment, with respect to the process for synthesis of compound 8 or 9, the “Step e” is performed at an ambient temperature.

In one embodiment, with respect to the process for synthesis of any of the compounds described herein, R³ of OR³ group or R⁵ of OR⁵ may be any conventional O-protecting group. Such O-protecting groups are described in T. W. Greene and P. G. M. Wuts, Protecting Groups in Organic Synthesis, Second Edition, Wiley, New York, 1991, and references cited therein. In specific embodiments each R³ or R⁵ are independently benzyl, THP, or alkanoyl groups. In further specific embodiments each R³ or R⁵ are independently any conventional silyl protecting groups. In yet further specific embodiments each R³ or R⁵ are independently TBS, TES, TMS, TBDMS, or TIPS groups.

In one embodiment, with respect to the process for synthesis of compound 8 or 9, the “Step f” is performed using chiral HPLC separation.

In another aspect of the invention discloses a use of any of the compounds described herein in synthesis of natural products.

In another aspect of the invention discloses a use of any of the compounds described herein in synthesis of any biologically active compounds.

In another aspect of the invention discloses a use of any of the compounds described herein in synthesis of any Resolvin derivatives.

In one embodiment, with respect to the use as described above, the compound is selected from 2-8, 10-13, 16-17, 19-23, 28-31, 36-38, 40, 42-45, 46-47, 53, 55-56, 58 and 59.

In another aspect of the invention discloses composition of any of the compounds disclosed herein. In one embodiment, the compounds are selected from 2-8, 10-13, 16-17, 19-23, 28-31, 36-38, 40, 42-45, 46-47, 53, 55-56, 58 and 59. In another embodiment, the compounds are not any of compounds 2-8, 10-13, 16-17, 19-23, 28-31, 36-38, 40, 42-45, 46-47, 53, 55-56, 58 and 59.

In one embodiment, the representative compounds as disclosed herein are named as given below:
Compound 5: 17-(tert-Butyl-dimethyl-silanyloxy)-docosa-4,7,10,13,15,19-hexenoic acid
Compound 6: 5-[13-(tert-Butyl-dimethyl-silanyloxy)-1-iodo-octadeca-3,6,9,11,15-pentaenyl]-dihydro-furan-2-one
Compound 7: 5-[13-(tert-Butyl-dimethyl-silanyloxy)-octadeca-1,3,6,9,11,15-hexaenyl]-dihydro-furan-2-one
Compound 8: 17-(tert-Butyl-dimethyl-silanyloxy)-4-hydroxy-docosa-5,7,10,13,15,19-hexaenoic acid
Compound 9: 17-(tert-Butyl-dimethyl-silanyloxy)-4-hydroxy-docosa-5,7,10,13,15,19-hexaenoic acid methyl ester
Compound 12: 4,17-Dihydroxy-docosa-5,7,10,13,15,19-hexaenoic acid methyl ester (Resolvin D6 methyl ester)
Compound 16: Docosa-4,7,10,13,16,19-hexaenoic acid (2-hydroxy-1-methyl-2-phenyl-ethyl)-methyl-amide
Compound 17: 5-(1-Iodo-octadeca-3,6,9,12,15-pentaenyl)-dihydro-furan-2-one
Compound 18: 5-Octadeca-1,3,6,9,12,15-hexaenyl-dihydro-furan-2-one
Compound 21: 4-Hydroxy-docosa-5,7,10,13,16,19-hexaenoic acid
Compound 22: 4-Hydroxy-docosa-5,7,10,13,16,19-hexaenoic acid methyl ester
Compound 23: 4-Oxo-docosa-5,7,10,13,16,19-hexaenoic acid methyl ester
Compound 24: 4(S)-Hydroxy-docosa-5,7,10,13,16,19-hexaenoic acid methyl ester
Compound 26: tert-Butyl-[1-(2-iodo-vinyl)-hex-3-enyloxy]-dimethyl-silane
Compound 27: 8-(tert-Butyl-dimethyl-silanyloxy)-1-trimethylsilanyl-trideca-6,10-diene-1,4-diyne
Compound 28: tert-Butyl-dimethyl-(1-pent-2-enyl-oct-2-ene-4,7-diynyloxy)-silane
Compound 29: 7-(tert-Butyl-dimethyl-silanyloxy)-9-iodo-nona-4,8-dienoic acid methyl ester
Compound 30: 7,17-Bis-(tert-butyl-dimethyl-silanyloxy)-docosa-4,8,15,19-tetraene-10,13-diynoic acid methyl ester
Compound 31: 7,17-Dihydroxy-docosa-4,8,15,19-tetraene-10,13-diynoic acid methyl ester
Compound 32: D4 Resolvin D5 methyl ester
Compound 33: D4 Resolvin D5

### General Synthetic Procedures

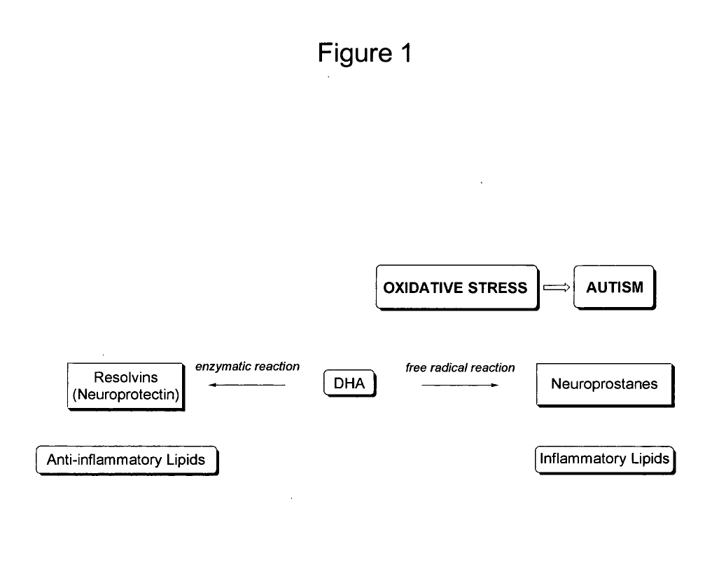

The compounds provided herein can be prepared from readily available starting materials using the following general methods and procedures. See, e.g., FIG. 1 and Synthetic Schemes 1-10 below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein, for example, may be prepared by the reaction of a chloro derivative with an appropriately substituted amine and the product isolated and purified by known standard procedures. Such procedures include (but are not limited to) recrystallization, column chromatography or HPLC. The following schemes are presented with details as to the preparation of representative fused heterocyclics that have been listed hereinabove. The compounds provided herein may be prepared from known or commercially available starting materials and reagents by one skilled in the art of organic synthesis.

The enantiomerically pure compounds provided herein may be prepared according to any techniques known to those of skill in the art. For instance, they may be prepared by chiral or asymmetric synthesis from a suitable optically pure precursor or obtained from a racemate by any conventional technique, for example, by chromatographic resolution using a chiral column, TLC or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

In certain embodiments, an enantiomerically pure compound of formula 1 may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the contents of which are hereby incorporated by reference in their entireties.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diasteroisomers can then be separated, for example, by chromatography or fractional crystallization, and the desired enantiomer regenerated by treatment with an appropriate base or acid. The other enantiomer may be obtained from the racemate in a similar manner or worked up from the liquors of the first separation.

In certain embodiments, enantiomerically pure compound can be separated from racemic compound by chiral chromatography. Various chiral columns and eluents for use in the separation of the enantiomers are available and suitable conditions for the separation can be empirically determined by methods known to one of skill in the art. Exemplary chiral columns available for use in the separation of the enantiomers provided herein include, but are not limited to CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

### General

All reactions that were moisture and air-sensitive were carried out in flame-dried glassware and under an argon atmosphere. The progress of the reactions was checked by thin layer chromatography (TLC) using E. Merck silica gel 60F glass plates (0.25 mm). The spots were visualized with UV light, followed by heat staining with p-anisaldehyde in EtOH/CH₃COOH/H₂SO₄. Silica gel 60 from EM-Science was used for flash chromatography. HPLC analysis were performed on a Hewlett-Packard liquid chromatograph HP-1090 Series II with PV5 SDS (Solvent Delivery System) and DAD (Diode Array Detector) equipped with heated column compartment and automatic liquid injector, or on a Waters HPLC system (M-6000A pump, M-730 Data Module integrator, U6K Injector) and a Schoeffel SF-770 UV detector. For Chiral HPLC Chiracel OF, OD, OB columns (Daicel Chemical Ltd.) have been used. ¹H NMR and ¹³C NMR data were recorded on a 300 MHz Varian Gemini 2000 Broadband High-Resolution NMR. IR spectra were measured on a Perkin-Elmer Paragon 1000 FT-IR spectrometer. UV Spectra were obtained using a Hewlett Packard HP-8453 UV-Visible Spectrophotometer. Optical rotation was measured on a Perkin-Elmer Polarimeter 343. Mass Spectra were obtained using Hewlett Packard HP-59987A API-Electrospray (Atmospheric Pressure Ionization Electrospray) interface coupled to a Mass Spectrometer Hewlett Packard HP-5989B MS. High Resolution Mass Spectra (HRMS) were obtained at the University of Pennsylvania Mass Spectrometry Service Center on a VG Micromass using Electrospray Ionization Mode. Microanalysis were performed by Micro-Analysis, Inc., Wilmington, Del.

In this specification, especially in “General Synthesis” and “Examples”, the following abbreviations can and may be used:
BEP 2-bromo-1-ethylpyridinium tetrafluoroborate
BOP benzotriazol-1-yloxy-tris(dimethylamino)phosphonium hexafluorophosphate
CDI 2-chloro-1,3-dimethylimidazolinium chloride
Co(TPP) 5, 10, 15, 20 tetraphenyl-21H, 23H porphine Co(II)
DCC dicyclohexylcarbodiimide
DCM dichloromethane
DME 1,2-dimethoxyethane, dimethoxyethane
DMF N,N-dimethylformamide
DMSO dimethyl sulfoxide
EDC 1-ethyl-3-(3′-dimethylaminopropyl)carbodiimide hydrogen chloride)
EtOAc ethyl acetate
EtOH ethanol
HBTU O-Benzotriazole-N,N,N′,N′-tetramethyl-uronium-hexafluoro-phosphate
HOBt 1-hydroxybenzotriazole
MeOH methanol
NMP N-methyl-2-pyrroliidone
PdCl₂ (pddf).CH₂Cl₂ palladiumdichloro-1,1′-bis(diphenylphosphino)ferrocene-dichloromethane complex
THF tetrahydrofuran
TFA trifluoroacetic acid
Cat: catalytic
PMN: Polymorphonuclear Neutrophil.
DHA: Docosahexaenoic acid (commonly known as DHA 22:6(ω-3), all-cis-docosa-4,7,10,13,16,19-hexaenoic acid.
DBU: 1,8-Diazabicyclo[5.4.0]undec-7-ene.
15-LOX: 15-Lipoxygenase
CBS-OXAZABOROLIDINE: general catalyst,
Methyl-CBS-oxazaborolidine: 3,3-Diphenyl-1-methylpyrrolidino[1,2-c]-1,3,2-oxazaborole
Ru—(S,S)-TsDPEN: Ruthenium-(1S,2S)-(+)-N-(4-toluenesulfonyl)-1,2-diphenylethylene-diamine catalyst
Ru—(R,R)-TsDPEN: Ruthenium-(1R,2R)-(−)-N-(4-toluenesulfonyl)-1,2-diphenylethylene-diamine catalyst
BINAL-H: Lithium Aluminium Hydride-2,2′-dihydroxy-1,1′-binaphthyl
TES: Triethylsilyl
TBS or TBDMS: t-Butyldimethylsilyl
TIPS: Triisopropylsilyl
TMS: Trimethylsilyl
(SS)-TSDPEN-Ru-(p-Cymene)-Cl₂: Ruthenium-(p-cymenc)-(1S,2S)-(+)-N-(4-toluene-sulfonyl)-1,2-diphenylethylenediamine dichloride
(RR)-TSDPEN-Ru-(p-Cymene)-Cl₂: Ruthenium-(p-cymene)-(1R,2R)-(+)-N-(4-toluene-sulfonyl)-1,2-diphenylethylenediamine dichloride

### EXAMPLES

### Example 1

5.3 g of the chiral phosphonium salt 53 was suspended in 60 ml anhydrous THF under Ar and cooled to −78 C. Then 25 ml of potassium bis(trimethylsilyl)amide (0.5 M solution in toluene, Aldrich) was slowly added and the mixture was stirred for 1 hour followed by addition of 1 g of the aldehyde ester 57. The reaction was stirred for 1 additional hour at −78 C and then warmed to 0 C over 1 hour. The mixture was put in 200 ml ether and washed with saturated ammonium chloride solution followed by NaCl solution. The aqueous phase was re-extracted with 2×100 ml ether, dried (sodium sulfate), and evaporated and purified by flash chromatography. Yield 58 (R6=Me):1.3 g.

### Example 2

1.6 g of the chiral isopropylidene ester 58 was dissolved in 120 ml methanol and cooled to 0 C. Then 16 ml 1N HCl was added and the reaction is stirred at 4 C overnight. Then 14 g solid NaHCO3 was added and the mixture was stirred at room temperature for 20 min, evaporated, extracted with ethyl acetate, dried (sodium sulfate) and purified by flash chromatography. R_{f}=0.54 (Ethyl acetate). Yield 58B (R⁶=Me):1.3 g.

### Example 3

To 1.3 g of the product 58B in 20 ml DMF at 0 C was added 4.2 g imidazole, 1.1 ml triethylamine followed by dropwise addition of 5 ml TES-Cl and stirred for 12 h at room temperature. The mixture was diluted with 200 ml ether and washed with sodium chloride solution 3×30 ml, dried (sodium sulfate), evaporate and purified by flash chromatography. Yield 59 (R⁶=Me): 2.8 g.

### Example 4

To a solution of 170 mg of bis(trimethylsilyl)acetylene 35 (R²=Me) in 10 ml of dichloromethane at 0 C was added 17.7 mg indium III bromide solid and stirred for 10 min, then 180 mg Methyl 4-chloro-4-oxo-butyrate 34 in 10 ml was slowly added and kept at 0 C for 3 hours. The reaction was quenched with ice water and re-extracted with 2×20 ml dichloromethane. Drying (sodium sulfate) and purification by flash chromatography provided the pure product. Yield 36 (R¹=R²=Me): 200 mg.

### Example 5

To a solution of 25 mg ((S,S)-TsDPEN)Ru(p-cymene)Cl₂ in 70 ml 2-propanol degassed under Argon and stirred for 20 min was slowly added 1.6 g 1-trimethylsilyl-1-pentyne-3-one 40 (R¹=R²=Me) in 20 ml degassed 2-propanol at room temperature over 5 hr. The reaction was complete as judged by TLC to give after flash chromatography 1.5 g of the chiral alcohol 40a with 99% ee as determined by chiral phase HPLC.

### Example 6

The diacetylene precursor of Resolvin D5 methyl ester 31 (R⁶=Me) (8 mg) was dissolved in 1 ml d4-methanol and 0.5 ml D₂O was added and then evaporated at room temperature to complete the HOD exchange in the reactant. In a two-neck flame dried flask was added freshly prepared Zn/Ag/Cu alloy (400 mg) which was suspended in 2 ml d4-methanol and 2 ml D2O. To this mixture was added 8 mg of the Resolvin D5 methyl ester precursor 31 (R⁶=Me) in 2 ml d4-methanol and stirred under argon at 40-45 C overnight. TLC and HPLC showed complete reaction. After the usual work up, the d4-Resolvin D5 methyl ester 32 (R⁶=Me) was purified by preparative HPLC.

### Example 7

### Synthesis of Compound 44

0.24 g of compound 43 (R¹=Me, R³=TBS) is added in a flame dried one necked flask under Argon, then 0.38 ml Tributyl tin hydride and 10 mg 2,2 Azobis(2-methylpropionitrile) AIBN is added and the flask is immersed in a preheated oil bath (130° C.). After a short period the reaction is started by the appearance of white fuming and stirred at the same temperature for 2 hours 30 min. After cooling to r.t. the mixture is purified by flash chromatography (Hexane/Ethyl acetate 95; 5) to yield 44 (R¹=Me, R³=TBS), 0.47 g.

### Example 8

### Synthesis of Compound 45

To a solution of 0.47 g of compound 44 (R¹=Me, R³=TBS) in Dichloromethane at −5 to −10° C. is added a dilute solution of Bromine in Dichloromethane until the yellow color persist. TLC showed total consumption of the starting material. The solution is concentrated and purified by flash chromatography (Hexane/Ethyl acetate 95; 5) to yield 45 (R¹=Me, R³=TBS) 0.47 g. Yield 0.173 g (60%) α²⁵_{D}-16 (c=0.98 CHCl₃).

Compounds 46 and 47 can be synthesized following the method described for compound 45.

### Example 9

### Synthesis of Compound 53

22 g of compound 52 was added under Argon in a 500 ml flask containing 7.6 g sodium bicarbonate and 28 g Triphenyl phosphine in 140 ml acetonitrile. The reaction is stirred for 70 hours at 50° C. and then evaporated at room temperature. To the oily residue is added 200 ml dry ether and the mixture upon slight shaking solidifies. The ether is decanted and the solid is washed twice with 100 ml dry ether and dried to give 53. Yield 38 g.

α²⁰_{D}+2 (c=0.37 CHCl₃) Lit+1.25 (CHCl₃).

### Example 10

### General Method for Hydrolysis of Lactones and Hydroxy methylesters to the Hydroxy Carboxyl Acids

### Compounds 18, 20, 32

To a solution of 20 (47 mg, 0.14 mmol) in THF (10 ml) at 0° C. under an argon atmosphere was added 1 N LiOH (2.8 ml, 2.8 mmol). The mixture was stirred for 2.5 h and diluted with EtOAc and layered with phosphate buffer pH 4.5. The phases were separated, the aqueous layer was re-extracted with EtOAc and the combined organic layers were dried (Na₂SO₄) and concentrated under vacuo affording the final compound.

### Example 11

### General Cleavage of O-Silyl Protective Groups

In a flamed dry flask under argon the starting material is dissolved in methanol and stirred at 0° C., then 20 ul of acetyl chloride is added and stirring is continued at the same temperature for 10 min and then at r.t. for 1-2 hr. TLC showed completion of the reaction. Addition of solid NaHCO₃ (100 mg) concentration and flash chromatography produced the desilylated products.

### Example 12

### General Method for Hydroxylation Using Lipoxylgenase

The synthesis of 17-Hydroxy docosahexaenoic acid using lipoxygenase biocatalyst was carried similar as described in Ref. 38.

### Example 12

### General Method for Iodolactonization

The iodolactonization was performed as described in the Lit. 39

### Example 13

### General Method for Amide Iodolactonization

The ephedrine Docosahexaenoic acid amide iodolactonization was performed as described by Myers et al., Ref. 53

### NMR Spectral Data:

Compound 3 (17OH-DCHA): ¹H NMR (CDCl₃, 300 MHz): δ 6.6-6.5 (ddt, J=15.3, 11.1, 1.2 Hz, 1H), 6.0 (br. t, J=11.1 Hz, 1H), 5.8-5.6 (dd, J=15.3, 6.0 Hz, 1H), 5.6-5.5 (dtt, J=10.8, 7.2, 1.5 Hz, 1H), 5.5-5.3 (m, 8H), 4.3-4.2 (m, 1H), 3.0-2.9 (m, 2H), 2.9-2.8 (m, 4H), 2.4-2.3 (m, 6H), 2.2-2.1 (m, 2H), 1.0-0.9 (t, J=7.5 Hz, 31-1); ¹³C NMR (CDCl₃, 75.5 MHz): δ 177.38, 135.53, 135.39, 130.43, 129.59, 128.62, 128.20 (2C), 128.11, 127.80 (2C), 125.61, 123.71, 72.05, 35.24, 33.77, 26.09, 25.66, 25.61, 22.64, 20.66, 14.02.

Compound 10 (17OH-DCHA-4-(R,S)-iodolactone): NMR (CDCl₃, 300 MHz): δ 6.6-6.5 (br. dd, J=15.0, 11.1 Hz, 1H), 6.0 (br. t, J=11.1 Hz, 1H), 5.8-5.6 (dd, J=15.0, 6.0 Hz, 1H), 5.6-5.5 (m, 2H), 5.5-5.3 (m, 5H), 4.3-4.2 (m, 2H), 4.1 (td, J=7.5, 2.7 Hz, 1H), 3.0-2.9 (m, 2H), 2.9-2.3 (m, 9H), 2.2-2.0 (m, 3H), 1.0-0.9 (t, J=7.5 Hz, 3H); ¹³C NMR (CDCl₃, 75.5 MHz): δ 176.21, 136.14, 135.21, 131.60, 129.99, 128.42, 128.33, 127.82, 126.92, 125.41, 123.93, 80.73, 71.95, 37.48 and 37.44, 35.33, 34.60 and 34.54, 28.42, 27.22 and 27.19, 26.15, 25.92, 20.67, 14.04.

Compound 11 (17OH-DCHA-γ-R,S-lactone): ¹H NMR (CDCl₃, 300 MHz): δ 6.7-6.5 (m, 2H), 6.0 (br. t, J=11.1 Hz, 2H), 5.8-5.6 (m, 2H), 5.6-5.3 (m, 6H), 5.1-5.0 (m, 1H), 4.2 (m, 1H), 3.0-2.9 (m, 4H), 2.6-2.5 (m, 2H), 2.5-2.3 (m, 3H), 2.2-2.0 (m, 3H), 1.9-1.8 (br. s, 1H), 1.0-0.9 (t, J=7.5 Hz, 3H); ¹³C NMR (CDCl₃, 75.5 MHz): δ 176.79, 136.17, 135.27, 132.38, 130.24, 129.93, 128.57, 128.36, 127.98 and 127.91, 127.70, 127.34, 125.38, 123.88, 80.34, 71.96, 35.37, 28.77, 28.40, 26.17, 26.11, 20.67, 14.04.

Compound 5 (17OTBS-DCHA): ¹H NMR (CDCl₃, 300 MHz): δ 6.6-6.5 (ddt, J=15.0, 11.1, 1.2 Hz, 1H), 6.0 (br. t, J=11.1 Hz, 1H), 5.7-5.6 (dd, J=15.0, 6.0 Hz, 1H), 5.5-5.3 (m, 9H), 4.2 (m, 1H), 3.0-2.9 (m, 2H), 2.9-2.7 (m, 4H), 2.4 (m, 4H), 2.3-2.2 (m, 2H), 2.0 (quint., J=7.2 Hz, 2H), 1.0-0.9 (t, J=7.5 Hz, 3H), 0.9 (s, 9H), 0.1-0.0 (2s, 6H); ¹³C NMR (CDCl₃, 75.5 MHz): δ 177.14, 137.02, 133.51, 129.67, 129.29, 128.47, 128.43, 128.36, 128.14, 128.08, 127.69, 124.79, 124.42, 73.21, 36.44, 33.72, 26.16, 25.94 (3C), 25.73, 25.68, 22.65, 20.77, 18.27, 14.11, −4.36, −4.65.

Compound 7 (17OTBS-DCHA-γ-lactone): ¹H NMR (CDCl₃, 300 MHz): δ 6.6 (dd, J=15.0, 11.1 Hz, 1H), 6.5-6.4 (dd, J=15.0, 11.1 Hz, 1H), 6.0 (m, 2H), 5.7-5.6 (m, 2H), 5.6-5.2 (m, 6H), 5.0 (m, 1H), 4.2 (m, 1H), 3.0-2.9 (m, 4H), 2.6-2.5 (m, 2H), 2.5-2.3 (m, 1H), 2.3-2.0 (m, 2H), 2.1-1.9 (m, 3H), 1.0-0.9 (t, J=7.5 Hz, 3H), 0.9 (s, 9H), 0.1-0.0 (2s, 6H); ¹³C NMR (CDCl₃, 75.5 MHz): δ 176.53, 137.18, 133.52, 132.40, 130.13, 128.94, 128.69, 128.53, 128.08, 127.57, 127.31, 124.71, 124.21, 80.43, 73.08, 36.39, 28.88, 28.52, 26.19, 26.14, 25.92 (3C), 20.75, 18.28, 14.13, −4.39, −4.66.

Compound 16 (DCHA-ephedrine-amide) (asterisk denotes minor rotamer peaks): ¹H NMR (CDCl₃, 300 MHz): δ 7.4-7.2 (m, 5H), 5.5-5.3 (m, 12H), 4.7-4.5 (m, 1H), 4.5-4.4 and (4.1-3.9)* (2m, 1H), 2.9* and 2.8 (2s, 3H), 2.9-2.8 (m, 10H), 2.5-2.2 (m, 4H), 2.2-2.0 (m, 2H), 1.1 and 1.0* (2d, J=6.9 Hz, 3H), 1.0-0.9 (t, J=7.5 Hz, 3H).

Compound 17 (5-iodo-DCHA-γ-lactone): ¹H NMR (CDCl₃, 300 MHz): δ 5.7-5.5 (m, 1H), 5.5-5.3 (m, 9H), 4.4-4.3 (td, J=7.2, 3.0 Hz, 1H), 4.2-4.1 (td, J=7.2, 3.0 Hz, 1H), 3.0-2.8 (m, 8H), 2.8-2.4 (m, 5H), 2.2-2.0 (m, 3H), 1.0 (t, J=7.5 Hz, 3H); ¹³C NMR (CDCl₃, 75.5 MHz): δ 176.04, 132.16, 131.69, 128.93, 128.74, 128.56, 128.04, 127.96, 127.47, 127.16, 126.86, 80.71, 37.42, 34.50, 28.40, 27.20, 25.87, 25.66, 25.63, 25.52, 20.48, 14.11.

Compound 18 (4-(S)-Zooxanthel-lactone): ¹H NMR (CDCl₃, 300 MHz): δ 6.6 (dd, J=15.3, 11.1 Hz, 1H), 6.0 (t, J=11.1 Hz, 1H), 5.7-5.6 (dd, J=15.3, 6.9 Hz, 1H), 5.6-5.4 (m, 1H), 5.5-5.2 (m, 8H), 5.0 (q, J=6.9 Hz, 1H), 3.0-2.9 (m, 2H), 2.9-2.7 (m, 6H), 2.6-2.5 (m, 2H), 2.5-2.3 (m, 1H), 2.1-1.9 (m, 3H), 1.0-0.9 (t, J=7.5 Hz, 3H); ¹³C NMR (CDCl₃, 75.5 MHz): δ 176.70, 132.48, 132.12, 130.15, 129.01, 128.71, 128.51, 128.06, 127.93, 127.89, 127.28, 127.25, 127.08, 80.41, 28.75, 28.41, 26.07, 25.59, 25.56, 25.46, 20.44, 14.08.

Compound 22: ¹H NMR (CDCl₃, 300 MHz): δ 6.6-6.4 (dd, J=15.3, 11.1 Hz, 1H), 6.0-5.9 (t, J=11.1 Hz, 1H), 5.7-5.6 (dd, J=15.3, 6.6 Hz, 1H), 5.5-5.2 (m, 9H), 4.3-4.1 (m, 1H), 3.6 (s, 3H), 3.0-2.9 (m, 2H), 2.9-2.7 (m, 6H), 2.5-2.4 (t, J=7.3 Hz, 2H), 2.2-2.0 (m, 2H), 1.9-1.8 (m, 2H), 1.0-0.9 (t, J=7.5 Hz, 3H); ¹³C NMR (CDCl₃, 75.5 MHz): δ 174.19, 135.53, 132.10, 130.67, 128.72, 128.69, 128.44, 128.02, 127.92 (2C), 127.63, 127.08, 125.88, 71.79, 51.59, 32.12, 30.09, 26.16, 25.73, 25.70, 25.59, 20.57, 14.20.

Compound 17: ¹H NMR (CDCl₃, 300 MHz): δ 7.6-7.4 (dd, J=15.3, 11.4 Hz, 1H), 6.2 (d, J=15.3 Hz, 1H), 6.1 (t, J=11.4 Hz, 1H), 5.9-5.8 (m, 1H), 5.5-5.2 (m, 8H), 3.7 (s, 3H), 3.1-3.0 (t, J=7.2 Hz, 2H), 2.9 (t, J=6.9 Hz, 2H), 2.9-2.7 (m, 6H), 2.6 (t, J=6.9 Hz, 2H), 2.1-2.0 (quint., J=7.5 Hz, 2H), 0.9 (t, J=7.5 Hz, 3H); ¹³C NMR (CDCl₃, 75.5 MHz): δ 197.93, 173.17, 139.88, 136.96, 132.09, 129.72, 129.54, 128.74, 128.72, 127.82, 127.69, 127.11, 127.08, 126.44, 51.62, 35.48, 28.04, 26.72, 25.77, 25.72, 25.60, 20.56, 14.15.

Compound 36 (4-Oxo-6-trimethylsilanyl-hex-5-ynoic acid methyl ester): ¹H NMR (CDCl₃, 300 MHz): δ 3.6 (s, 3H), 2.9-2.8 (t, J=6.6 Hz, 2H), 2.6 (t, J=6.6 Hz, 2H), 0.2 (s, 9H); ¹³C NMR (CDCl₃, 75.5 MHz): δ 185.06, 172.38, 101.45, 98.61, 51.79, 39.82, 27.65, −0.87.

Compound 38 (5-Oxo-7-trimethylsilanyl-hept-6-ynoic acid methyl ester): ¹H NMR (CDCl₃, 300 MHz): δ 3.7 (s, 3H), 2.7-2.6 (t, J=6.6 Hz, 2H), 2.4-2.3 (t, J=6.6 Hz, 2H), 2.0-1.9 (quint., J=6.6 Hz, 2H), 0.2 (s, 9H); ¹³C NMR (CDCl₃, 75.5 MHz): δ 186.71, 173.38, 101.93, 98.13, 51.47, 44.12, 32.76, 18.94, −0.94.

Compound 25 (1-Trimethylsilanyl-pent-1-yn-3-one): ¹H NMR (CDCl₃, 300 MHz): δ 2.6-2.5 (q, J=7.5 Hz, 2H), 1.1 (t, J=7.5 Hz, 3H), 0.2 (s, 91-1); ¹³C NMR (CDCl₃, 75.5 MHz): δ 188.10, 101.91, 97.55, 38.54, 7.89, −0.82.

Compound 40 (4-Hydroxy-6-trimethylsilanyl-hex-5-ynoic acid methyl ester): ¹H NMR (CDCl₃, 300 MHz): δ 4.5-4.4 (m, 1H), 3.6 (s, 3H), 2.6-2.4 (m, 3H), 2.1-1.9 (m, 2H), 0.1 (s, 9H); ¹³C NMR (CDCl₃, 75.5 MHz): δ 173.94, 105.74, 89.98, 61.80, 51.67, 32.39, 29.68, −0.22.

Compound 42 (4-(tert-Butyl-dimethyl-silanyloxy)-6-trimethylsilanyl-hex-5-ynoic acid methyl ester): ¹H NMR (CDCl₃, 300 MHz): δ 4.4 (t, J=6.0 Hz, 1H), 3.6 (s, 3H), 2.4 (t, J=7.5 Hz, 2H), 2.0-1.8 (m, 2H), 0.9 (s, 9H), 0.12 (s, 9H), 0.10 (s, 3H), 0.07 (s, 3H); ¹³C NMR (CDCl₃, 75.5 MHz): δ 173.66, 106.88, 89.32, 62.37, 51.39, 33.51, 29.66, 25.81 (3C), 18.22, −0.22, −4.51, −4.97.

Compound 43 (4-(tert-Butyl-dimethyl-silanyloxy)-hex-5-ynoic acid methyl ester): ¹H NMR (CDCl₃, 300 MHz): δ 4.4 (td, J=6.0 Hz, 1H), 3.6 (s, 3H), 2.5 (t, J=7.5 Hz, 2H), 2.4 (d, J=1.8 Hz, 1H), 2.1-1.9 (m, 2H), 0.9 (s, 9H), 0.10 (s, 3H), 0.07 (s, 3H); ¹³C NMR (CDCl₃, 75.5 MHz): δ 173.61, 84.67, 72.58, 61.61, 51.48, 33.41, 29.40, 25.71 (3C), 18.14, −4.69, −5.18.

Compound 44 (4-(tert-Butyl-dimethyl-silanyloxy)-6-tributylstannanyl-hex-5-enoic acid methyl ester): ¹H NMR (CDCl₃, 300 MHz): δ 6.6-6.0 (d, J=19.2 Hz, 1H), 6.0-5.8 (dd, J=19.2, 5.7 Hz, 1H), 4.1-4.0 (m, 1H), 3.6 (s, 3H), 2.4-2.2 (t, J=7.5 Hz, 2H), 1.8-1.7 (m, 2H), 1.6-1.4 (quint., J=7.5 Hz, 6H), 1.4-1.2 (hex., J=7.5 Hz, 6H), 0.9-0.8 (m, 15H), 0.9 (s, 9H), 0.01 (s, 3H), 0.0 (s, 3H); ¹³C NMR (CDCl₃, 75.5 MHz): δ 174.16, 150.96, 127.87, 75.44, 51.30, 32.91, 29.74, 29.14 (3C), 27.21 (3C), 25.93 (3C), 18.27, 13.58 (3C), 9.60 (3C), −4.31, −4.83.

Compound 45 (6-Bromo-4-(tert-butyl-dimethyl-silanyloxy)-hex-5-enoic acid methyl ester): ¹H NMR (CDCl₃, 300 MHz): δ 6.3-6.2 (d, J=13.5 Hz, 1H), 6.2-6.1 (dd, J=13.5, 5.7 Hz, 1H), 4.2 (q, J=5.7 Hz, 1H), 3.6 (s, 3H), 2.4-2.3 (t, J=7.5 Hz, 2H), 1.9-1.7 (m, 2H), 0.9 (s, 9H), 0.1-0.0 (2s, 6H); ¹³C NMR (CDCl₃, 75.5 MHz): δ 173.67, 140.13, 106.53, 71.95, 51.46, 32.54, 29.13, 25.81 (3C), 18.15, −4.54, −4.94.

Compound 32 (d₄-Resolvin-D5 methyl ester): ¹H NMR (CD₃CN, 300 MHz): δ 6.6 (d, J=15.3 Hz, 2H), 5.8-5.7 (dd, J=15.3, 6.0 Hz, 2H), 5.6-5.3 (m, 4H), 4.2-4.1 (m, 2H), 3.6 (s, 3H), 3.1 (s, 2H), 2.6-2.2 (m, 8H), 2.2-2.0 (m, 2H), 1.0-0.9 (t, J=7.5 Hz, 3H); ¹³C NMR (CD₃CN, 75.5 MHz)(CD due to its intensity are not observed): δ 174.00, 137.89, 137.80, 134.22, 130.52, 127.38, 125.39, 125.22, 125.18, 72.14, 72.00, 51.59, 35.90, 35.87, 34.20, 26.62, 23.41, 21.06, 14.13.

Compound 56: ¹³C NMR (CDCl₃, 75.5 MHz): 133.33, 125.14, 73.49, 66.90, 32.36, 20.70, 14.10, 6.79, 6.70, 6.57, 5.20, 4.59.

Compound 27: ¹³C NMR (CDCl₃, 75.5 MHz): 145.93, 133.91, 124.10, 108.75, 99.82, 85.18, 83.25, 79.13, 72.61, 35.97, 25.87, 20.73, 18.20, 14.05, 11.54, −0.10, −4.55, −4.77.

Compound 28: ¹³C NMR (CDCl₃, 75.5 MHz): 146.18, 133.93, 124.09, 108.55, 82.83, 79.37, 78.15, 72.59, 68.70, 35.97, 25.88, 20.74, 14.05, 10.23, −4.55, −4.76.

Compound 30: ¹³C NMR (CDCl₃, 75.5 MHz): C-1 not observed (weak signal) 145.88, 145.63, 133.82, 127.67, 126.36, 124.07, 108.94, 108.70, 83.75, 83.53, 79.08, 78.98, 72.66, 72.44, 51.43, 36.08, 34.08, 25.07, 23.19, 20.85, 18.32, 14.16, 11.24, −4.38, −4.61.

Compound 31: ¹³C NMR (CD₃CN, 75.5 MHz): 173.99, 147.08, 146.98, 134.90, 130.90, 126.92, 124.93, 109.26, 109.19, 84.53, 84.45, 79.46, 79.44, 71.69, 71.53, 51.62, 35.43, 34.18, 23.41, 21.07, 14.11, 10.95.

Compound 32: (D4 Resolvin D5 methyl ester)

UV (λ max 244 nm ethanol) (ε: 33.500), HPLC/API-MS (m/z) 402 [M+Na+]+

Compound 33: (D4 Resolvin D5)

UV (λ max 244 nm ethanol) (ε: 33.500), HPLC/API-MS (m/z) 388 [M+Na+]+

The syntheses of representative compounds of this invention can be carried out in accordance with the methods set forth above and using the appropriate reagents, starting materials, and purification methods known to those skilled in the art.

### REFERENCES

1. Schwab J M, Serhan C N; Lipoxins and new lipid mediators in the resolution of inflammation, CURRENT OPINION IN PHARMACOLOGY 6 (4): 414-420 August 2006
2. Arita M, Oh S F, Chonan T, et al.; Metabolic inactivation of resolvin E1 and stabilization of its anti-inflammatory actions, JOURNAL OF BIOLOGICAL CHEMISTRY 281 (32): 22847-22854 Aug. 11, 2006
3. Hudert C A, Weylandt K H, Lu Y, et al.; Transgenic mice rich in endogenous ω-3 fatty acids are protected from colitis, PROCEEDINGS OF THE NATIONAL ACADEMY OF SCIENCES OF THE UNITED STATES OF AMERICA 103 (30): 11276-11281 Jul. 25, 2006
4. Chonan T, Arita M, Takayama K, et al.; Scale up synthesis of resolvin E1 and its bioaction, ABSTRACTS OF PAPERS OF THE AMERICAN CHEMICAL SOCIETY 230: U2584-U2585 132-MEDI Aug. 28, 2005
5. Arita M, Bianchini F, Aliberti J, et al.; Omega-3 derived lipid mediator resolvin E1: Stereochemistry, receptor, and anti-inflammatory and protective actions in colitis PROSTAGLANDINS & OTHER LIPID MEDIATORS 79 (1-2): 154-154 March 2006
6. Serhan C N, Gotlinger K, Hong S, et al.; Anti-inflammatory actions of neuroprotectin D1/protectin D1 and its natural stereoisomers: Assignments of dihydroxy-containing docosatrienes, JOURNAL OF IMMUNOLOGY 176 (3): 1848-1859 Feb. 1, 2006
7. Hasturk H, Kantarci A, Ohira T, et al.; RvE1 protects from local inflammation and osteoclast-mediated bone destruction in periodontitis, FASEB JOURNAL 19 (14): 401+ December 2005
8. Hong S, Tjonahen E, Morgan E L, et al.; Rainbow trout (Oncorhynchus mykiss) brain cells biosynthesize novel docosahexaenoic acid-derived resolvins and protectins—Mediator lipidornic analysis, PROSTAGLANDINS & OTHER LIPID MEDIATORS 78 (1-4): 107-116 December 2005
9. Arita M, Clish C B, Serhan C N; The contributions of aspirin and microbial oxygenase to the biosynthesis of anti-inflammatory resolvins: Novel oxygenase products from omega-3 polyunsaturated fatty acids, BIOCHEMICAL AND BIOPHYSICAL RESEARCH COMMUNICATIONS 338 (1): 149-157 Dec. 9, 2005
10. Weylandt K H, Kang J X; Rethinking lipid mediators, LANCET 366 (9486): 618-620 Aug. 20, 2005
11. Arita M, Yoshida M, Hong S, et al.; Resolvin E1, an endogenous lipid mediator derived from omega-3 eicosapentaenoic acid, protects against 2,4,6-trinitrobenzene sulfonic acid-induced colitis, PROCEEDINGS OF THE NATIONAL ACADEMY OF SCIENCES OF THE UNITED STATES OF AMERICA 102 (21): 7671-7676 May 24, 2005
12. Bannenberg G L, Chiang N, Ariel A, et al.; Molecular circuits of resolution: Formation and actions of resolvins and protectins, JOURNAL OF IMMUNOLOGY 174 (7): 4345-4355 Apr. 1, 2005
13. Flower R J, Perretti M; Controlling inflammation: a fat chance? JOURNAL OF EXPERIMENTAL MEDICINE 201 (5): 671-674 Mar. 7, 2005
14. Arita M, Bianchini F, Aliberti J, et al.; Stereochemical assignment, antiinflammatory properties, and receptor for the omega-3 lipid mediator resolvin E1 JOURNAL OF EXPERIMENTAL MEDICINE 201 (5): 713-722 Mar. 7, 2005
15. Serhan C N, Arita M, Hong S, et al.; Resolvins, docosatrienes, and neuroprotectins, novel omega-3-derived mediators, and their endogenous aspirin-triggered epimers LIPIDS 39 (11): 1125-1132 November 2004
16. Serhan C N, Gotlinger K, Hong S, et al.; Resolvins, docosatrienes, and neuroprotectins, novel omega-3-derived mediators, and their aspirin-triggered endogenous epimers: an overview of their protective roles in catabasis, PROSTAGLANDINS & OTHER LIPID MEDIATORS 73 (3-4): 155-172 April 2004
17. Adibhatla R M, Hatcher J F, Dempsey R J; Lipids and lipidomics in brain injury and diseases, AAPS JOURNAL 8 (2): E314-E321 2006
18. Serhan C N, Gotlinger K, Hong S, et al.; Anti-inflammatory actions of neuroprotectin D1/protectin D1 and its natural stereoisomers: assignments of dihydroxy-containing docosatrienes. (vol 176, pg 1848, 2006), JOURNAL OF IMMUNOLOGY 176 (6): 3843-3843 Mar. 15, 2006
19. Bazan N G; Cell survival matters: docosahexaenoic acid signaling, neuroprotection and photoreceptors, TRENDS IN NEUROSCIENCES 29 (5): 263-271 May 2006
20. Bazan N G; Survival signaling in retinal pigment epithelial cells in response to oxidative stress: Significance in retinal degenerations, ADVANCES IN EXPERIMENTAL MEDICINE AND BIOLOGY 572: 531-540 2006
21. Butovich I A; A one-step method of 10,17-dihydro(pero)xydocosahexa-4Z,7Z,11E,13Z,15E,19Z-enoic acid synthesis by soybean lipoxygenase JOURNAL OF LIPID RESEARCH 47 (4): 854-863 April 2006
22. Bazan N G, Marcheselli V L, Cole-Edwards K; Brain response to injury and neurodegeneration—Endogenous neuroprotective signaling ANNALS OF THE NEW YORK ACADEMY OF SCIENCES 1053: 137-147 2005
23. Serhan C N, Gotlinger K, Hong S, et al.; Anti-inflammatory actions of neuroprotectin D1/protectin D1 and its natural stereoisomers: Assignments of dihydroxy-containing docosatrienes, JOURNAL OF IMMUNOLOGY 176 (3): 1848-1859 Feb. 1, 2006
24. Ariel A, Li P L, Wang W, et al.; The docosatriene Protectin D1 is produced by T(H)2 skewing and promotes human T cell apoptosis via lipid raft clustering JOURNAL OF BIOLOGICAL CHEMISTRY 280 (52): 43079-43086 Dec. 30, 2005
25. Hong S, Tjonahen E, Morgan E L, et al.; Rainbow trout (Oncorhynchus mykiss) brain cells biosynthesize novel docosahexaenoic acid-derived resolvins and protectins—Mediator lipidornic analysis, PROSTAGLANDINS & OTHER LIPID MEDIATORS 78 (1-4): 107-116 December 2005
26. Butovich I A; On the structure and synthesis of neuroprotectin D1, a novel anti-inflammatory compound of the docosahexaenoic acid family JOURNAL OF LIPID RESEARCH 46 (11): 2311-2314 November 2005
27. Lukiw W J, Cui J G, Marcheselli V L, et al.; A role for docosahexaenoic acid-derived neuroprotectin D1 in neural cell survival and Alzheimer disease, JOURNAL OF CLINICAL INVESTIGATION 115 (10): 2774-2783 October 2005
28. Chen C, Bazan N G; Lipid signaling: Sleep, synaptic plasticity, and neuroprotection PROSTAGLANDINS & OTHER LIPID MEDIATORS 77 (1-4): 65-76 September 2005
29. Bazan N G; Lipid signaling in neural plasticity, brain repair, and neuroprotection MOLECULAR NEUROBIOLOGY 32 (1): 89-103 August 2005
30. Bazan N G, Marcheselli V L, Mukherjee P K, et al.; Neurotrophic growth factors up-regulate neuroprotectin D1 (NPD1) synthesis and antiapoptotic signaling in human retinal pigment epithelial (RPE) cells, JOURNAL OF NEUROCHEMISTRY 94: 32-32 Suppl. 1 Jun. 2005
31. Bazan N G; Synaptic signaling by lipids in the life and death of neurons MOLECULAR NEUROBIOLOGY 31 (1-3): 219-230 2005
32. Gronert K, Maheshwari N, Khan N, et al.; A role for the mouse 12/15-lipoxygenase pathway in promoting epithelial wound healing and host defense JOURNAL OF BIOLOGICAL CHEMISTRY 280 (15): 15267-15278 Apr. 15, 2005
33. Bazan N G; Neuroprotectin D1 (NPD1): A DHA-derived mediator that protects brain and retina against cell injury-induced oxidative stress, BRAIN PATHOLOGY 15 (2): 159-166 April 2005
34. Serhan C N, Gotlinger K, Hong S, et al.; Resolvins, docosatrienes, and neuroprotectins, novel omega-3-derived mediators, and their aspirin-triggered endogenous epimers: an overview of their protective roles in catabasis, PROSTAGLANDINS & OTHER LIPID MEDIATORS 73 (3-4): 155-172 April 2004
35. Mukherjee P K, Marcheselli V L, Serhan C N, et al.; Neuroprotectin D1: A docosahexaenoic acid-derived docosatriene protects human retinal pigment epithelial cells from oxidative stress, PROCEEDINGS OF THE NATIONAL ACADEMY OF SCIENCES OF THE UNITED STATES OF AMERICA 101 (22): 8491-8496 Jun. 1, 2004
36. Rodriguez A R, Spur B W; First total synthesis of 7(S),16(R),17(S)-Resolvin D2, a potent anti-inflammatory lipid mediator, TETRAHEDRON LETTERS 45 (47): 8717-8720 Nov. 15, 2004
37. Rodriguez A R, Spur B W; First total synthesis of 7(S),17(S)-resolvin D5, a potent anti-inflammatory docosanoid, TETRAHEDRON LETTERS 46 (21): 3623-3627 May 23, 2005
38. Nanda S, Yadav J S; Lipoxygenase biocatalysis: a survey of asymmetric oxygenation JOURNAL OF MOLECULAR CATALYSIS B-ENZYMATIC 26 (1-2): 3-28 Nov. 3, 2003
39. COREY E J, HASHIMOTO S I; A PRACTICAL PROCESS FOR LARGE-SCALE SYNTHESIS OF (S)-5-HYDROXY-6-TRANS-8,11,14-CIS-EICOSATETRAENOIC ACID (5-HETE), TETRAHEDRON LETTERS 22 (4): 299-302 1981
40. COREY E J, ALBRIGHT J O, BARTON A E, et al.; CHEMICAL AND ENZYMIC SYNTHESES OF 5-HPETE—KEY BIOLOGICAL PRECURSOR OF SLOW-REACTING SUBSTANCE OF ANAPHYLAXIS (SRS), AND 5-HETE JOURNAL OF THE AMERICAN CHEMICAL SOCIETY 102 (4): 1435-1436 1980
41. COREY E J, SU W G; SIMPLE SYNTHESIS AND ASSIGNMENT OF STEREOCHEMISTRY OF LIPOXIN-A, TETRAHEDRON LETTERS 26 (3): 281-284 1985
42. COREY E J, MEHROTRA M M, SU W G; ON THE SYNTHESIS AND STRUCTURE OF LIPOXIN-B, TETRAHEDRON LETTERS 26 (16): 1919-1922 1985
43. COREY E J, SU W G, CLEAVER MB; A SIMPLE AND EFFICIENT SYNTHESIS OF (7E,9E,11Z,13E)-(5S,6R,15S)-TRIHYDROXYEICOSATETRAENOIC ACID (6R-LIPOXIN A), TETRAHEDRON LETTERS 30 (32): 4181-4184 1989
44. Haas J, Bissmire S, Wirth T; Iodine monochloride-amine complexes: An experimental and computational approach to new chiral electrophiles, CHEMISTRY-A EUROPEAN JOURNAL 11 (19): 5777-5785 Sep. 19, 2005
45. Wang M, Gao L X, Mai W P, et al.; Enantioselective iodolactonization catalyzed by chiral quaternary ammonium salts derived from cinchonidine, JOURNAL OF ORGANIC CHEMISTRY 69 (8): 2874-2876 Apr. 16, 2004
46. Wang M, Gao L X, Yue W, et al.; Reagent-controlled asymmetric iodolactonization using cinchona alkaloids as chiral sources, SYNTHETIC COMMUNICATIONS 34 (6): 1023-1032 March 2004
47. Blot V, Reboul V, Metzner P; Asymmetric induction of the Iodolactonization reaction of alpha-sulfurated gamma-unsaturated amides, JOURNAL OF ORGANIC CHEMISTRY 69 (4): 1196-1201 Feb. 20, 2004
48. Price M D, Kurth M J, Schore N E; Comparison of solid-phase and solution-phase chiral auxiliaries in the alkylation/Iodolactonization sequence to gamma-butyrolactones JOURNAL OF ORGANIC CHEMISTRY 67 (22): 7769-7773 Nov. 1, 2002
49. Grossman R B, Trupp R J; The first reagent-controlled asymmetric halolactonizations. Dihydroquinidine-halogen complexes as chiral sources of positive halogen ion, CANADIAN JOURNAL OF CHEMISTRY-REVUE CANADIENNE DE CHIMIE 76 (9): 1233-1237 September 1998
50. MOON H, SCHORE N E, KURTH M J; A Polymer-Supported C-2-Symmetrical Chiral Auxiliary—Preparation Of Non-Racemic 3,5-Disubstituted-Gamma-Butyrolactones, TETRAHEDRON LETTERS 35 (48): 8915-8918 Nov. 28, 1994
51. MOON H S, EISENBERG S W E, WILSON M E, et al.; Preparation Of Nonracemic 3,5-Disubstituted-Gamma-Butyrolactones—An Effective Sequent Auxiliary For Amide Alkylation And Iodolactonization, JOURNAL OF ORGANIC CHEMISTRY 59 (22): 6504-6505 Nov. 4, 1994
52. HOFFMANN R W, STURMER R, HARMS K; A Phenyl-Assisted Asymmetric Iodolactonization Reaction, TETRAHEDRON LETTERS 35 (34): 6263-6266 Aug. 22, 1994
53. Myers A G, McKinstry L; Practical syntheses of enantiomerically enriched gamma-lactones and gamma-hydroxy ketones by the alkylation of pseudoephedrine amides with epoxides and their derivatives, JOURNAL OF ORGANIC CHEMISTRY 61 (7): 2428-2440 Apr. 5, 1996
54. ONODERA K; Zooxanthellactone, a novel gamma-lactone-type oxylipine from dinoflagellates of Symbiodinium sp.: Structure, distribution, and biological activity BIOSCIENCE BIOTECHNOLOGY AND BIOCHEMISTRY 68: 848 2004
55. COREY E J, BAKSHI R K, SHIBATA S, et al.; A Stable And Easily Prepared Catalyst For The Enantioselective Reduction Of Ketones—Applications To Multistep Syntheses, JOURNAL OF THE AMERICAN CHEMICAL SOCIETY 109 (25): 7925-7926 Dec. 9, 1987
56. COREY E J, BAKSHI R K, SHIBATA S; Highly Enantioselective Borane Reduction Of Ketones Catalyzed By Chiral Oxazaborolidines—Mechanism And Synthetic Implications, JOURNAL OF THE AMERICAN CHEMICAL SOCIETY 109 (18): 5551-5553 Sep. 2, 1987
57. Rodriguez A, Nomen M, Spur B W, et al. An efficient asymmetric synthesis of prostaglandin E-1 EUROPEAN JOURNAL OF ORGANIC CHEMISTRY (10): 2655-2662 October 1999
58. van Bergen M, Gais H J; A new strategy for the enantioselective synthesis of carba-prostacyclin analogues based on organocopper conjugate addition to a bicyclic azoene and its application to the synthesis of 13,14-dinor-inter-p-phenylene carbacyclin, JOURNAL OF THE AMERICAN CHEMICAL SOCIETY 124 (16): 4321-4328 Apr. 24, 2002
59. Kramp G J, Kim M, Gais H J, et al.; Fully stereocontrolled total syntheses of the prostacyclin analogues 16S-iloprost and 16S-3-oxa-iloprost by a common route, using alkenylcopper-azoalkene conjugate addition, asymmetric olefination, and allylic alkylation, JOURNAL OF THE AMERICAN CHEMICAL SOCIETY 127 (50): 17910-17920 Dec. 21, 2005
60. Yu C M, Kim C, Kweon J H, Enantioselective synthesis of allenyl carbinols by the CBS reduction in nitroethane: dramatic solvent effect for reactivity and enantioselectivity CHEMICAL COMMUNICATIONS (21): 2494-2495 2004
61. Cho B T, Shin S H, Highly enantioselective synthesis of multifunctionalized allylic building blocks via oxazaborolidine-catalyzed borane reduction TETRAHEDRON 61 (29): 6959-6966 Jul. 18, 2005
62. Cho B T; Recent advances in the synthetic applications of the oxazaborolidine-mediated asymmetric reduction, TETRAHEDRON 62 (33): 7621-7643 Aug. 14, 2006
63. Kim M, Gais H J; Fully stereocontrolled syntheses of 3-oxacarbacyclin and carbacyclin by the conjugate addition-azoalkene-asymmetric olefination strategy JOURNAL OF ORGANIC CHEMISTRY 71 (12): 4642-4650 Jun. 9, 2006
64. Noyori R, Hashiguchi S; Asymmetric transfer hydrogenation catalyzed by chiral ruthenium complexes, ACCOUNTS OF CHEMICAL RESEARCH 30 (2): 97-102 February 1997
65. Noyori R; Asymmetric catalysis: Science and opportunities (Nobel lecture) ANGEWANDTE CHEMIE-INTERNATIONAL EDITION 41 (12): 2008-2022 2002
66. Ohkuma T, Utsumi N, Tsutsumi K, et al.; The hydrogenation/transfer hydrogenation network: Asymmetric hydrogenation of ketones with chiral eta(6)-arene/N-tosylethylenediamine-ruthenium(II) catalysts, JOURNAL OF THE AMERICAN CHEMICAL SOCIETY 128 (27): 8724-8725 Jul. 12, 2006
67. Yamakawa M, Yamada I, Noyori R; CH/pi attraction: The origin of enantioselectivity in transfer hydrogenation of aromatic carbonyl compounds catalyzed by chiral eta(6)-arene-ruthenium(II) complexes, ANGEWANDTE CHEMIE-INTERNATIONAL EDITION 40 (15): 2818-2821 2001
68. Matsumura K, Hashiguchi S, Ikariya T, et al.; Asymmetric transfer hydrogenation of alpha,beta-acetylenic ketones, JOURNAL OF THE AMERICAN CHEMICAL SOCIETY 119 (37): 8738-8739 Sep. 17, 1997
69. Haack K J, Hashiguchi S, Fujii A, et al.; The catalyst precursor, catalyst, and intermediate in the Ru-II-promoted asymmetric hydrogen transfer between alcohols and ketones ANGEWANDTE CHEMIE-INTERNATIONAL EDITION IN ENGLISH 36 (3): 285-288 Feb. 14, 1997
70. Wisman R V, de Vries J G, Deelman B J, et al.; Kinetic studies on the asymmetric transfer hydrogenation of acetophenone using a homogeneous ruthenium catalyst with a chiral amino-alcohol ligand, ORGANIC PROCESS RESEARCH & DEVELOPMENT 10 (3): 423-429 May 2006
71. Marshall J A, Ellis K; Applications of chiral allenylzinc additions and Noyori asymmetric reductions to an enantioselective synthesis of a C3-C13 precursor of the polyketide phosphatase inhibitor cytostatin, TETRAHEDRON LETTERS 45 (7): 1351-1353 Feb. 9, 2004
72. Marshall J A, Bourbeau M P; Synthesis of enantioenriched propargylic alcohols related to polyketide natural products. A comparison of methodologies, ORGANIC LETTERS 5 (18): 3197-3199 Sep. 4, 2003
73. Everaere K, Mortreux A, Carpentier J F; Rutheuium(II)-catalyzed asymmetric transfer hydrogenation of carbonyl compounds with 2-propanol and ephedrine-type ligands ADVANCED SYNTHESIS & CATALYSIS 345 (1-2): 67-77 January 2003
74. Yadav J S, Reddy B V S, Reddy M S, et al.; InBr3-catalyzed alkynylation and allylation of acid chlorides: A facile synthesis of alkynyl and allyl ketones, SYNTHESIS-STUTTGART (15): 2390-2394 Oct. 31, 2003
75. Taber D F, Xu M, Hartnett J C; Synthesis of the eight enantiomerically pure diastereomers of the 12-F-2-isoprostanes, JOURNAL OF THE AMERICAN CHEMICAL SOCIETY 124 (44): 13121-13126 Nov. 6, 2002.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the invention as given and set forth in this application, may have been generated on an automated basis by use of a commercially available chemical naming software program, and have not been independently verified. Representative programs performing this function include ChemDraw. In the instance where the indicated chemical name and the depicted structure differ, the depicted structure will control.

Chemical structures shown herein were prepared using ISIS®/DRAW. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A process for the synthesis of the compound of structure 1: 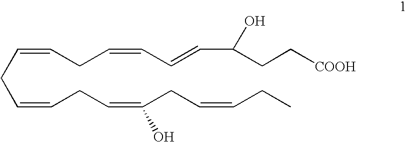
comprising the steps of:
a) converting the acid 2 to compound 3: 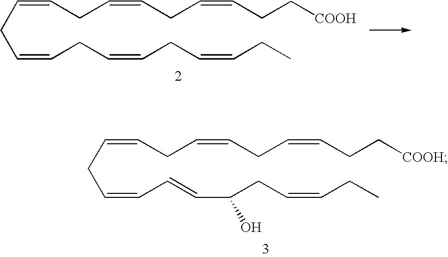
b) converting the compound 3 to compound 10: 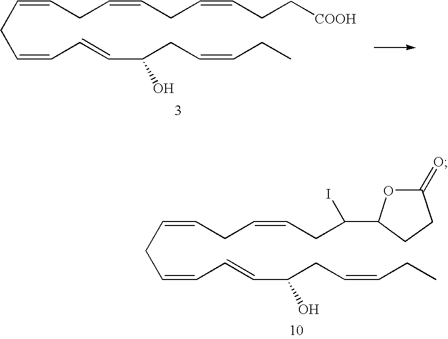
c) converting the compound 10 to compound 11: 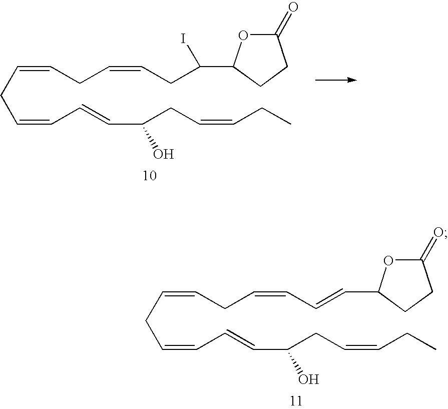
and
d) hydrolyzing compound 11 to the compound 1:

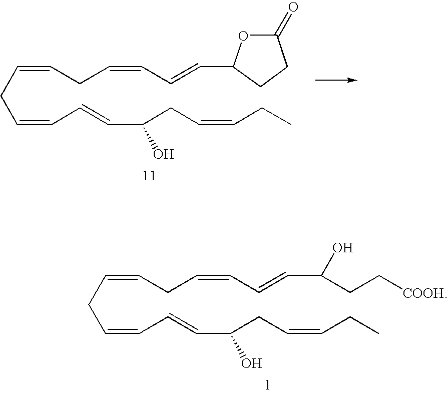

2. A compound selected from the group consisting of:

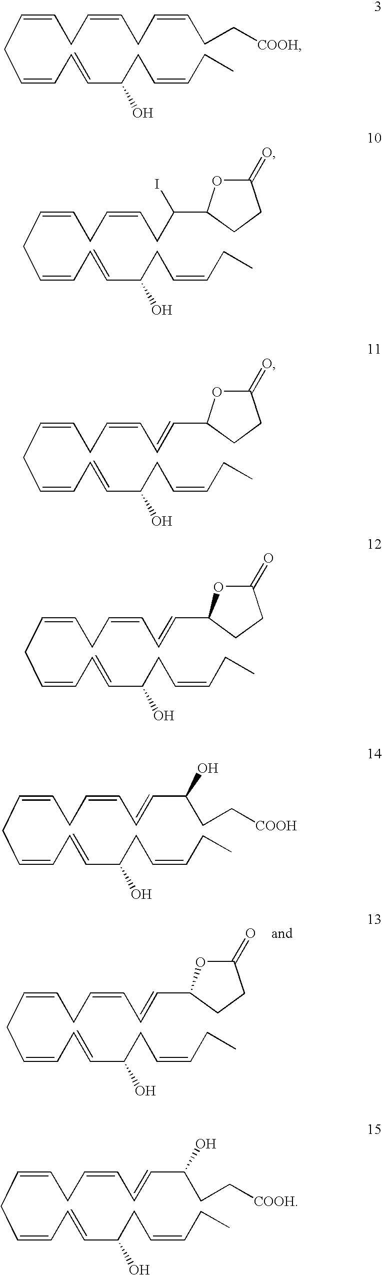

3. A process according to claim 1 wherein “Step a” comprises:
a) lipoxygenase oxidation; and then
b) reduction of the intermediate hydroperoxide.

4. A process according to claim 3 wherein the lipoxygenase oxidation is carried out in the presence of a buffer.

5. (canceled)

6. A process according to claim 3 wherein the lipoxygenase oxidation is carried out at between pH 8-10.

7. (canceled)

8. A process according to claim 3 wherein the reduction is carried out in the presence of a PPh₃.

9. A process according to claim 1 wherein “Step b” comprises:
a) iodolactonization.

10. A process according to claim 9 wherein the iodolactonization is carried out in the presence of
a. I₂;
b. KI or NaI;
c. a base; and
d. a solvent.

11. (canceled)

12. (canceled)

13. (canceled)

14. A process according to claim 1 wherein “Step c” comprises:
a) elimination.

15. A process according to claim 14 wherein the elimination is carried out in the presence of a base.

16. (canceled)

17. A process according to claim 1 wherein “Step c” is carried out at 15-25° C.

18. A process according to claim 1 wherein “Step d” comprises:
a) reaction with a base; and then
b) reaction with an acid.

19. A process according to claim 18 wherein the base is selected from LiOH, KOH and NaOH.

20. (canceled)

21. A process according to claim 18 wherein the reaction with a base is carried out in THF:H₂O mixture.

22. A process according to claim 18 wherein the acid is an acid capable of generating pH 4.5.

23. (canceled)

24. A process according to claim 1 wherein the “Step d” is carried out at 0-45° C.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A process for the synthesis of the compound of structure 24: 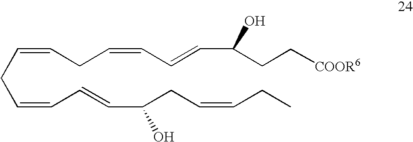
comprising the steps of:
a) converting the acid 2 to compound 19: 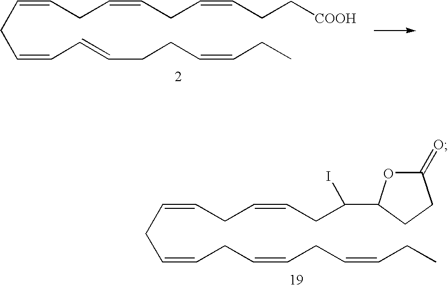
b) converting the compound 19 to compound 20: 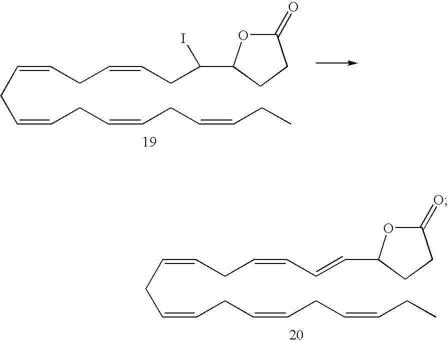
c) converting the compound 20 to compound 21: 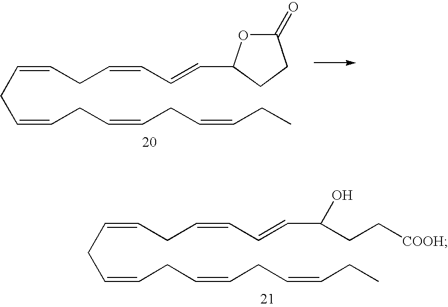
d) converting the compound 21 to compound 22: 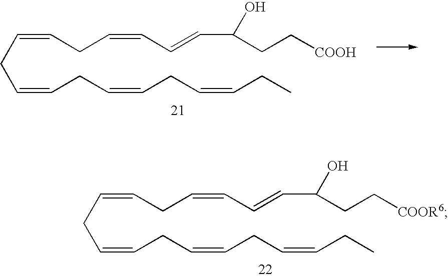
e) converting the compound 22 to compound 23: 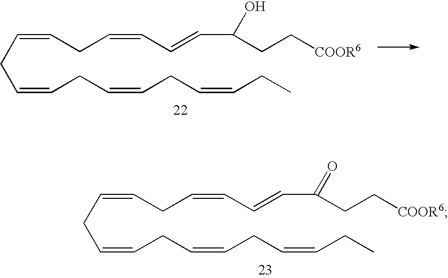
and
f) reducing the compound 23 to the compound 24: 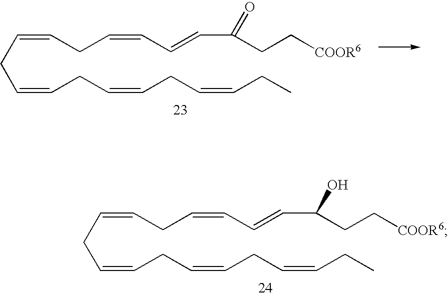
wherein R⁶ is alkyl or benzyl.

44. A process according to claim 43 wherein R⁶ is Me, Et, n-Bu, benzyl, or t-Bu.

45. (canceled)

46. A process for the synthesis of the compound of structure 33: 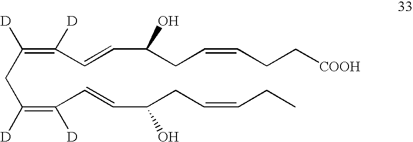
comprising the steps of:
a) condensing compound 25 with compound 26 to afford compound 27: 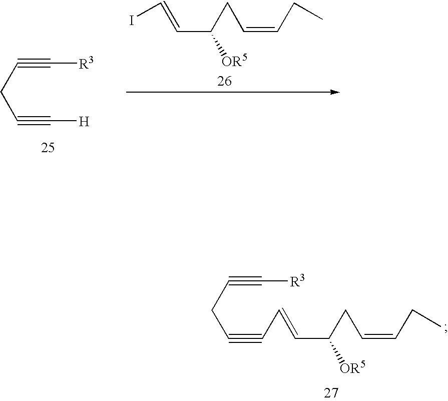
b) converting the compound 27 to compound 28: 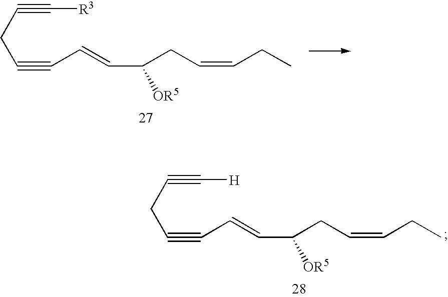
c) coupling of the compound 28 with compound 29 to give compound 30: 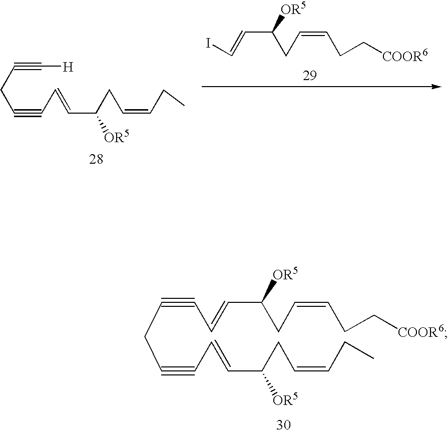
d) deprotecting compound 30 to the compound 31: 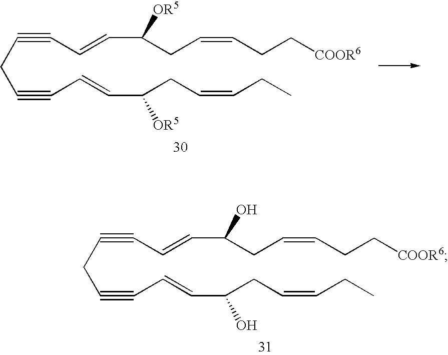
e) converting compound 31 to the compound 32: 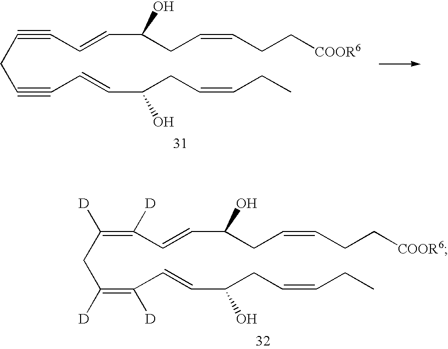
and
f) converting compound 32 to the compound 33: 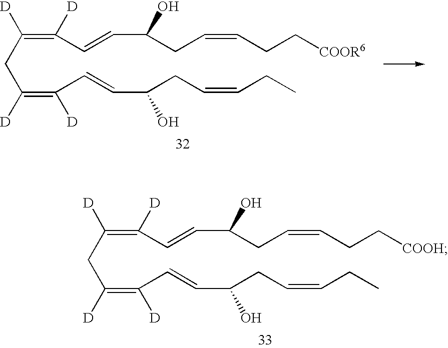
wherein each R³ is silyl protecting group; and R⁵ is silyl or O-protecting group; and R⁶ is alkyl or benzyl.

47. A process according to claim 46 wherein R³ is TMS, TIPS, TBDMS, TBS, or TES.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)
